(19) United States
(12) Patent Application Publication
(10) Pub. No.: US 2024/0261363 A1
(43) Pub. Date: Aug. 8, 2024

(54) METHODS OF TREATING AND PREVENTING CANCER TREATMENT SIDE EFFECTS

(71) Applicant: FILAMENT BIOSOLUTIONS INC., New York, NY (US)

(72) Inventors: Thomas P. CIRRITO, Long Island City, NY (US); Kate M. Rochlin, New York, NY (US)

(21) Appl. No.: 18/483,874

(22) Filed: Oct. 10, 2023

Related U.S. Application Data
(63) Continuation of application No. 16/321,921, filed on Jan. 30, 2019, now abandoned.
(60) Provisional application No. 62/369,424, filed on Aug. 1, 2016.

(51) Int. Cl. A61K 38/05; A61K 9/00; A61K 33/30; A61K 45/06; A61K 47/18; A61P 35/00; C07C 231/24; C07C 237/06; C07K 5/00; C07K 5/062

(52) U.S. Cl. CPC A61K 38/05; A61K 9/0053; A61K 33/30; A61K 47/183; A61P 35/00; C07C 231/24; C07C 237/06; C07K 5/00; C07K 5/06026; A61K 45/06

(57) ABSTRACT

Provided herein are Alanyl-Glutamine (Ala-Gin) formulations for use in treating and preventing side effects associated with cancer treatment. Also provided herein are methods of using the formulations provided herein to treat or prevent side effects associated with cancer treatment.

### 1. INTRODUCTION

Provided herein are Alanyl-Glutamine (Ala-Gln) formulations for use in treating and preventing side effects associated with cancer treatment. Also provided herein are methods of using the formulations provided herein to treat or prevent side effects associated with cancer treatment.

### 2. BACKGROUND

Chemotherapy and radiation represent the most common therapies for the treatment of cancers. Typically, both chemotherapy and radiation are administered to cancer patients at the highest possible tolerated dosage in order to achieve maximum effectiveness in eradicating cancer cells. However, the inherent ability of these treatments to eradicate cells through targeting cell growth and replication factors frequently leads to damage in non-cancerous cells and organs, resulting in multiple side-effects and toxicities. The incidence of these side effects can be extremely high and, when severe enough, the side effects become dose-limiting, i.e., the dosage must be reduced or modified due to patient pain or discomfort, or because the side effects themselves become life threatening. The result of such dose-limiting side effects is suboptimal treatment of patients, because the full course and/or intensity of chemotherapy and/or radiation cannot be administered. Accordingly, there is a need for methods of reducing or eliminating side effects known to accompany cancer treatment methods such as chemotherapy and radiation.

### 3. SUMMARY

In one aspect, provided herein are Alanyl-Glutamine formulations for use in treating and preventing side effects associated with cancer treatment, e.g., side effects caused by
therapy and/or radiation. The Alanyl-Glutamine formulations provided herein can be formulated for oral administration, e.g., as a beverage, and can comprise one or more additional components. In a specific embodiment, said Alanyl-Glutamine formulations further comprise zinc.

In a specific embodiment, the Alanyl-Glutamine formulations provided herein comprise Alanyl-Glutamine in an appropriate solution (e.g., water). Such formulations can, in certain embodiments, comprise about 1 gram of Alanyl-Glutamine/ounce of solution, about 5 grams of Alanyl-Glutamine/ounce of solution, about 10 grams of Alanyl-Glutamine/ounce of solution, about 15 grams of Alanyl-Glutamine/ounce of solution, about 20 grams of Alanyl-Glutamine/ounce of solution, about 25 grams of Alanyl-Glutamine/ounce of solution, about 30 grams of Alanyl-Glutamine/ounce of solution, about 35 grams of Alanyl-Glutamine/ounce of solution, about 40 grams of Alanyl-Glutamine/ounce of solution, about 45 grams of Alanyl-Glutamine/ounce of solution, about 50 grams of Alanyl-Glutamine/ounce of solution, about 55 grams of Alanyl-Glutamine/ounce of solution, about 60 grams of Alanyl-Glutamine/ounce of solution, about 65 grams of Alanyl-Glutamine/ounce of solution, about 70 grams of Alanyl-Glutamine/ounce of solution, about 75 grams of Alanyl-Glutamine/ounce of solution, about 80 grams of Alanyl-Glutamine/ounce of solution, about 85 grams of Alanyl-Glutamine/ounce of solution, about 90 grams of Alanyl-Glutamine/ounce of solution, about 95 grams of Alanyl-Glutamine/ounce of solution, or about 100 grams of Alanyl-Glutamine/ounce of solution.

In a specific embodiment, the Alanyl-Glutamine formulations provided herein comprise about 1-5 grams of Alanyl-Glutamine/ounce of solution, about 5-10 grams of Alanyl-Glutamine/ounce of solution, about 10-15 grams of Alanyl-Glutamine/ounce of solution, about 15-20 grams of Alanyl-Glutamine/ounce of solution, about 20-25 grams of Alanyl-Glutamine/ounce of solution, about 25-30 grams of Alanyl-Glutamine/ounce of solution, about 30-35 grams of Alanyl-Glutamine/ounce of solution, about 35-40 grams of Alanyl-Glutamine/ounce of solution, about 40-45 grams of Alanyl-Glutamine/ounce of solution, about 45-50 grams of Alanyl-Glutamine/ounce of solution, about 50-55 grams of Alanyl-Glutamine/ounce of solution, about 55-60 grams of Alanyl-Glutamine/ounce of solution, about 60-65 grams of Alanyl-Glutamine/ounce of solution, about 65-70 grams of Alanyl-Glutamine/ounce of solution, about 70-75 grams of Alanyl-Glutamine/ounce of solution, about 75-80 grams of Alanyl-Glutamine/ounce of solution, about 80-85 grams of Alanyl-Glutamine/ounce of solution, about 85-90 grams of Alanyl-Glutamine/ounce of solution, about 90-95 grams of Alanyl-Glutamine/ounce of solution, or about 95-100 grams of Alanyl-Glutamine/ounce of solution.

In another specific embodiment, an Alanyl-Glutamine formulation provided herein comprises about 1-100 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 5-60 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 10-30 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 20-30 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 10 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 14.8 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 15 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 19.7 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 20 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 22.2 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 30 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 44.4 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 59.2 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In certain embodiments, the Alanyl-Glutamine formulations provided herein, including any of the foregoing formulations, comprise zinc (e.g., zinc from zinc sulfate monohydrate or heptahydrate). In a specific embodiment, said formulations comprise about 1 mg zinc/ounce of solution, about 5 mg zinc/ounce of solution, about 10 mg zinc/ounce of solution, about 15 mg zinc/ounce of solution, about 20 mg zinc/ounce of solution, about 25 mg zinc/ounce of solution, about 30 mg zinc/ounce of solution, about 35 mg zinc/ounce of solution, about 40 mg zinc/ounce of solution, about 45 mg zinc/ounce of solution, about 50 mg zinc/ounce of solution, about 55 mg zinc/ounce of solution, about 60 mg zinc/ounce of solution, about 65 mg zinc/ounce of solution, about 70 mg zinc/ounce of solution, about 75 mg zinc/ounce of solution, about 80 mg zinc/ounce of solution, about 85 mg zinc/ounce of solution, about 90 mg zinc/ounce of solution, about 95 mg zinc/ounce of solution, about 100 mg zinc/ounce of solution, about 125 mg zinc/ounce of solution, about 150 mg zinc/ounce of solution, about 175 mg zinc/ounce of solution, about 200 mg zinc/ounce of solution, about 250 mg zinc/ounce of solution, about 300 mg zinc/ounce of solution, about 400 mg zinc/ounce of solution, or about 500 mg zinc/ounce of solution.

In certain embodiments, the Alanyl-Glutamine formulations provided herein, including any of the foregoing formulations, comprise about 1-5 mg zinc/ounce of solution, about 5-10 mg zinc/ounce of solution, about 10-15 mg zinc/ounce of solution, about 15-20 mg zinc/ounce of solution, about 20-25 mg zinc/ounce of solution, about 25-30 mg zinc/ounce of solution, about 30-35 mg zinc/ounce of solution, about 35-40 mg zinc/ounce of solution, about 40-45 mg zinc/ounce of solution, about 45-50 mg zinc/ounce of solution, about 50-55 mg zinc/ounce of solution, about 55-60 mg zinc/ounce of solution, about 60-65 mg zinc/ounce of solution, about 65-70 mg zinc/ounce of solution, about 70-75 mg zinc/ounce of solution, about 75-80 mg zinc/ounce of solution, about 80-85 mg zinc/ounce of solution, about 85-90 mg zinc/ounce of solution, about 90-95 mg zinc/ounce of solution, about 95-100 mg zinc/ounce of solution, about 100-125 mg zinc/ounce of solution, about 125-150 mg zinc/ounce of solution, about 150-175 mg zinc/ounce of solution, about 175-200 mg zinc/ounce of solution, about 200-250 mg zinc/ounce of solution, about 250-300 mg zinc/ounce of solution, about 300-400 mg zinc/ounce of solution, or about 400-500 mg zinc/ounce of solution.

In a specific embodiment, an Alanyl-Glutamine formulation provided herein comprises about 1-100 grams of Alanyl-Glutamine and about 10-200 mg of zinc in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In a specific embodiment, an Alanyl-Glutamine formulation provided herein comprises about 5-60 grams of Alanyl-Glutamine and about 10-100 mg of zinc in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In a specific embodiment, an Alanyl-Glutamine formulation provided herein comprises about 10-30 grams of Alanyl-Glutamine and about 10-50 mg of zinc in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In certain embodiments, the Alanyl-Glutamine formulations provided herein can comprise one or more of the following additional components: sweeteners (e.g., glucose, maltodextrin, maltose, dextrose, high fructose corn syrup, sucrose, lactose, fructose, Saccharin, aspartame, acesulfame K, sucralose, neotame, rebaudioside A, Stevia/Erythritol, Sorbitol, Mannitol, and Xylitol); thickening and/or gelling agents (e.g., alginate, propylene glycol alginate, carrageenans, cellulose gum and microcrystalline cellulose, Xanthum gums, and guar gums), coloring agents (e.g., Blue No. 2 (Indigotine), Red No. 40 (Allura Red AC), Yellow No. 5 (Tartrazine)); flavor and fragrance agents; agents capable of maintaining the formulations at a desired pH (e g, citric acid, sodium citrate, zinc sulphase, magnesium sulphate, potassium chloride, and magnesium oxide); vitamins (e.g., vitamins A, D, E, K, B, C, Niacin, and folic acids); masking agents (e.g., citric acid, sodium citrates, sodium phosphate, and hydroxyl acids); and/or bitter taste-receptor blockers (e.g., hydrogenated ethoxylated glycerol ester, Beta-Cyclodextrin, homoeriodictyol sodium salt, and eriodictyol).

In certain embodiments, the Alanyl-Glutamine formulations provided herein are present in a container (e.g., a bottle, flask, tube, jar) that can be provided to a subject in need of ingestion of an Alanyl-Glutamine formulation, e.g., a patient undergoing cancer treatment or that is scheduled to undergo a cancer treatment. In a specific embodiment, a single container comprising an Alanyl-Glutamine formulation comprises a single dose of Alanyl-Glutamine. In another specific embodiment, a single container comprising an Alanyl-Glutamine formulation comprises multiple doses of Alanyl-Glutamine, e.g., 1, 2, 3, 4, 5, 6, 7, 8, 9, or 10 doses of Alanyl-Glutamine.

In another aspect, provided herein are methods of treating or preventing side effects associated with cancer treatment, wherein said methods comprise oral administration of an Alanyl-Glutamine formulation described herein. In a specific embodiment, the Alanyl-Glutamine formulation comprises zinc. In another specific embodiment, the Alanyl-Glutamine formulation is administered as a beverage.

In a specific embodiment, the methods of treating or preventing side effects associated with cancer treatment provided herein treat or prevent side effects caused by administration of chemotherapy.

In another specific embodiment, the methods of treating or preventing side effects associated with cancer treatment provided herein treat or prevent side effects caused by administration of radiation.

In another specific embodiment, the side effect caused by cancer treatment that is treated or prevented by the methods provided herein is oral mucositis. See Section 5.2.3.1.

In another specific embodiment, the side effect caused by cancer treatment that is treated or prevented by the methods provided herein is peripheral neuropathy. See Section 5.2.3.2.

In another specific embodiment, the side effect caused by cancer treatment that is treated or prevented by the methods provided herein is cardiac damage, e.g., cardiomyopathy. See Section 5.2.3.3.

In another specific embodiment, the side effect caused by cancer treatment that is treated or prevented by the methods provided herein is dysgeusia. See Section 5.2.3.4.

In another specific embodiment, the side effect caused by cancer treatment that is treated or prevented by the methods provided herein is hypertension. See Section 5.2.3.5.

In certain embodiments of the methods of treating or preventing side effects associated with cancer treatment provided herein, a patient diagnosed with cancer is administered an Alanyl-Glutamine formulation described herein prior to initiation of a cancer treatment regimen, e.g., before administration of chemotherapy and/or radiation. In a specific embodiment, the patient is administered an Alanyl-Glutamine formulation described herein 1, 2, 3, or 4 weeks prior to initiation of a cancer treatment regimen. In another specific embodiment, the patient is administered an Alanyl-Glutamine formulation described herein 1, 2, 3, 4, 5, 6, 7, 8, 9, 10, 11, 12, 13, 14, 15, 16, 17, 18, 19, 20, or 21 days prior to initiation of a cancer treatment regimen. In another specific embodiment, the patient is administered an Alanyl-Glutamine formulation described herein on the same day that a cancer treatment regimen begins, but before a cancer treatment is administered to the patient. In another specific embodiment, once a patient has first been administered an Alanyl-Glutamine formulation described herein (day 1), they are administered an Alanyl-Glutamine formulation described herein daily for the course of the cancer treatment. In a specific embodiment, the side effect caused by cancer treatment that is treated or prevented is oral mucositis, peripheral neuropathy, cardiac damage (e.g., cardiomyopathy), dysgeusia, and/or hypertension.

In certain embodiments of the methods of treating or preventing side effects associated with cancer treatment provided herein, a patient diagnosed with cancer is administered an Alanyl-Glutamine formulation described herein after initiation of a cancer treatment regimen, e.g., after administration of chemotherapy and/or radiation. In a specific embodiment, the patient is administered an Alanyl-Glutamine formulation described herein 1, 2, 3, or 4 weeks after initiation of a cancer treatment regimen. In another specific embodiment, the patient is administered an Alanyl-Glutamine formulation described herein 1, 2, 3, 4, 5, 6, 7, 8, 9, 10, 11, 12, 13, 14, 15, 16, 17, 18, 19, 20, or 21 days after initiation of a cancer treatment regimen. In another specific embodiment, the patient is administered an Alanyl-Glutamine formulation described herein on the same day that a cancer treatment regimen begins, but after a cancer treatment is administered to the patient. In another specific embodiment, once a patient has first been administered an Alanyl-Glutamine formulation described herein (day 1), they are administered an Alanyl-Glutamine formulation described herein daily for the course of the cancer treatment. In a specific embodiment, the side effect caused by cancer treatment that is treated or prevented is oral mucositis, peripheral neuropathy, cardiac damage (e.g., cardiomyopathy), dysgeusia, and/or hypertension.

In certain embodiments of the methods of treating or preventing side effects associated with cancer treatment provided herein, a patient diagnosed with cancer is administered an Alanyl-Glutamine formulation described herein for the duration of the cancer treatment, e.g., as long as the patient is being treated for cancer (e.g., being administered chemotherapy or radiation, or both), the patient is administered an Alanyl-Glutamine formulation described herein. In a specific embodiment, the patient is administered an Alanyl-Glutamine formulation described herein daily for the course of the cancer treatment. In a specific embodiment, the side effect caused by cancer treatment that is treated or prevented is oral mucositis, peripheral neuropathy, cardiac damage (e.g., cardiomyopathy), dysgeusia, and/or hypertension.

In certain embodiments of the methods of treating or preventing side effects associated with cancer treatment provided herein, a patient diagnosed with cancer is administered an Alanyl-Glutamine formulation described herein after a cancer treatment has been completed, e.g., the patient is administered an Alanyl-Glutamine formulation described herein for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after the cancer treatment has been completed (e.g., after the final dose of chemotherapy and/or radiation); for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks after the cancer treatment has been completed (e.g., after the final dose of chemotherapy and/or radiation); or for 1, 2, 3, 4, or 5 years after the cancer treatment has been completed (e.g., after the final dose of chemotherapy and/or radiation). In a specific embodiment, the patient is administered an Alanyl-Glutamine formulation described herein daily after the cancer treatment has been completed. In a specific embodiment, the side effect caused by cancer treatment that is treated or prevented is oral mucositis, peripheral neuropathy, cardiac damage (e.g., cardiomyopathy), dysgeusia, and/or hypertension.

In a specific embodiment of the methods of treating or preventing side effects associated with cancer treatment provided herein, a patient diagnosed with cancer is administered an Alanyl-Glutamine formulation described herein (i) prior to initiation of a cancer treatment regimen (e.g., before administration of chemotherapy and/or radiation), for example, 1, 2, 3, 4, 5, 6, 7, 8, 9, 10, 11, 12, 13, 14, 15, 16, 17, 18, 19, 20, or 21 days prior to initiation of a cancer treatment regimen; or 1, 2, 3, or 4 weeks prior to initiation of a cancer treatment regimen; and (ii) for the duration of the cancer treatment, e.g., as long as the patient is being treated for cancer (e.g., being administered chemotherapy or radiation, or both). In a specific embodiment, the side effect caused by cancer treatment that is treated or prevented is oral mucositis, peripheral neuropathy, cardiac damage (e.g., cardiomyopathy), dysgeusia, and/or hypertension.

In another specific embodiment of the methods of treating or preventing side effects associated with cancer treatment provided herein, a patient diagnosed with cancer is administered an Alanyl-Glutamine formulation described herein (i) prior to initiation of a cancer treatment regimen (e.g., before administration of chemotherapy and/or radiation), for example, 1, 2, 3, 4, 5, 6, 7, 8, 9, 10, 11, 12, 13, 14, 15, 16, 17, 18, 19, 20, or 21 days prior to initiation of a cancer treatment regimen; or 1, 2, 3, or 4 weeks prior to initiation of a cancer treatment regimen; (ii) for the duration of the cancer treatment, e.g., as long as the patient is being treated for cancer (e.g., being administered chemotherapy or radiation, or both); and (iii) after the cancer treatment has been completed (e.g., after the final dose of chemotherapy and/or radiation), e.g., for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after the cancer treatment has been completed; for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks after the cancer treatment has been completed; or for 1, 2, 3, 4, or 5 years after the cancer treatment has been completed. In a specific embodiment, the side effect caused by cancer treatment that is treated or prevented is oral mucositis, peripheral neuropathy, cardiac damage (e.g., cardiomyopathy), dysgeusia, and/or hypertension.

In accordance with the methods of treating or preventing side effects associated with cancer treatment provided herein, it is to be understood that administration of an Alanyl-Glutamine formulation described herein to a patient in need thereof, e.g., a subject undergoing or about to undergo a cancer treatment (e.g., chemotherapy or radiation) is performed in a manner that results in the patient receiving a dose of Alanyl-Glutamine. A dose of Alanyl-Glutamine can be administered as part of a single administration or as part of multiple administrations, wherein said multiple administrations can occur over the course of 1, 2, 3, 4, 5, 6, or 7 days. In a specific embodiment, said multiple administrations occur over the course of 1 day.

In certain embodiments, a dose of Alanyl-Glutamine is administered over the course of 1 day on a daily basis as part of a method described herein, wherein the daily dose is divided over the course of 2, 3, 4, 5, 6, 7, 8, 9, or 10 administrations of an Alanyl-Glutamine formulation described herein. In a specific embodiment, a dose of Alanyl-Glutamine is administered on a daily basis as part of a method described herein, wherein the daily dose is divided over the course of 2 administrations of an Alanyl-Glutamine formulation described herein, i.e., a patient is administered an Alanyl-Glutamine formulation described herein twice a day. In another specific embodiment, a dose of Alanyl-Glutamine is administered on a daily basis as part of a method described herein, wherein the daily dose is divided over the course of 3 administrations of an Alanyl-Glutamine formulation described herein, i.e., a patient is administered an Alanyl-Glutamine formulation described herein three times a day. In another specific embodiment, a dose of Alanyl-Glutamine is administered on a daily basis as part of a method described herein, wherein the daily dose is divided over the course of 4 administrations of an Alanyl-Glutamine formulation described herein, i.e., a patient is administered an Alanyl-Glutamine formulation described herein four times a day. In another specific embodiment, a dose of Alanyl-Glutamine is administered on a daily basis as part of a method described herein, wherein the daily dose is divided over the course of 5 administrations of an Alanyl-Glutamine formulation described herein, i.e., a patient is administered an Alanyl-Glutamine formulation described herein five times a day.

In certain embodiments, a dose of Alanyl-Glutamine is administered on a daily basis as part of a method described herein, wherein the daily dose is not divided over the course of multiple administrations, but instead is administered as part of a single administration, i.e., a patient is administered an Alanyl-Glutamine formulation described herein once a day.

In certain embodiments, a single dose of Alanyl-Glutamine as used in accordance with the methods provided herein comprises about 5 grams of Alanyl-Glutamine, about 10 grams of Alanyl-Glutamine, about 15 grams of Alanyl-Glutamine, about 20 grams of Alanyl-Glutamine/ounce of solution, about 25 grams of Alanyl-Glutamine, about 30 grams of Alanyl-Glutamine, about 35 grams of Alanyl-Glutamine, about 40 grams of Alanyl-Glutamine, about 45 grams of Alanyl-Glutamine, about 50 grams of Alanyl-Glutamine, about 55 grams of Alanyl-Glutamine, about 60 grams of Alanyl-Glutamine, about 65 grams of Alanyl-Glutamine, about 70 grams of Alanyl-Glutamine, about 75 grams of Alanyl-Glutamine, about 80 grams of Alanyl-Glutamine, about 85 grams of Alanyl-Glutamine, about 90 grams of Alanyl-Glutamine, about 95 grams of Alanyl-Glutamine, or about 100 grams of Alanyl-Glutamine. In a specific embodiment, said single dose of Alanyl-Glutamine is administered with zinc (e.g., a solution that comprises both Alanyl-Glutamine and zinc is administered or a solution that comprises Alanyl-Glutamine and a separate solution that comprises zinc are administered), wherein about 10, 15, 20, 25, 30, 35, 40, 45, 50, 55, 60, 65, 70, 75, 80, 85, 90, 95, 100, 125, 150, 175, 200, 225, 250, 300, 350, 400, 450, or 500 mg of zinc is administered with said dose of Alanyl-Glutamine. In another specific embodiment, said single dose of Alanyl-Glutamine is administered with zinc (e.g., a solution that comprises both Alanyl-Glutamine and zinc is administered or a solution that comprises Alanyl-Glutamine and a separate solution that comprises zinc are administered), wherein about 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-125, 125-150, 150-175, 175-200, 200-250, 250-300, 300-350, 350-400, 400-450, or 450-500 mg of zinc is administered with said dose of Alanyl-Glutamine. In certain embodiments, said dose of Alanyl-Glutamine or said dose of Alanyl-Glutamine and zinc is administered over the course of 1 day in one or multiple (e.g., 2, 3, 4, or 5) administrations.

In certain embodiments, a single dose of Alanyl-Glutamine as used in accordance with the methods provided herein comprises about 5-10 grams of Alanyl-Glutamine, about 10-15 grams of Alanyl-Glutamine, about 15-20 grams of Alanyl-Glutamine, about 20-25 grams of Alanyl-Glutamine, about 25-30 grams of Alanyl-Glutamine, about 30-35 grams of Alanyl-Glutamine, about 35-40 grams of Alanyl-Glutamine about 40-45 grams of Alanyl-Glutamine, about 45-50 grams of Alanyl-Glutamine, about 50-55 grams of Alanyl-Glutamine, about 55-60 grams of Alanyl-Glutamine, about 60-65 grams of Alanyl-Glutamine, about 65-70 grams of Alanyl-Glutamine, about 70-75 grams of Alanyl-Glutamine, about 75-80 grams of Alanyl-Glutamine, about 80-85 grams of Alanyl-Glutamine, about 85-90 grams of Alanyl-Glutamine, about 90-95 grams of Alanyl-Glutamine, or about 95-100 grams of Alanyl-Glutamine. In a specific embodiment, said single dose of Alanyl-Glutamine is administered with zinc (e.g., a solution that comprises both Alanyl-Glutamine and zinc is administered or a solution that comprises Alanyl-Glutamine and a separate solution that comprises zinc are administered), wherein about 10, 15, 20, 25, 30, 35, 40, 45, 50, 55, 60, 65, 70, 75, 80, 85, 90, 95, 100, 125, 150, 175, 200, 225, or 250, 300, 350, 400, 450, or 500 mg of zinc is administered with said dose of Alanyl-Glutamine. In another specific embodiment, said single dose of Alanyl-Glutamine is administered with zinc (e.g., a solution that comprises both Alanyl-Glutamine and zinc is administered or a solution that comprises Alanyl-Glutamine and a separate solution that comprises zinc are administered), wherein about 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-125, 125-150, 150-175, 175-200, 200-250, 250-300, 300-350, 350-400, 400-450, or 450-500 mg of zinc is administered with said dose of Alanyl-Glutamine. In certain embodiments, said dose of Alanyl-Glutamine or said dose of Alanyl-Glutamine and zinc is administered over the course of 1 day in one or multiple (e.g., 2, 3, 4, or 5) administrations.

In a specific embodiment, a single dose of Alanyl-Glutamine as used in accordance with the methods provided herein comprises about 20-75 grams of Alanyl-Glutamine. In another specific embodiment, said dose of Alanyl-Glutamine as used in accordance with the methods is administered with zinc, e.g., 100-400 mg of zinc. In certain embodiments, said dose is administered over the course of 1 day in one or multiple (e.g., 2, 3, 4, or 5) administrations.

In another specific embodiment, a single dose of Alanyl-Glutamine as used in accordance with the methods provided herein comprises about 20-60 grams of Alanyl-Glutamine. In another specific embodiment, said dose of Alanyl-Glutamine as used in accordance with the methods is administered with zinc, e.g., 100-400 mg of zinc. In certain embodiments, said dose is administered over the course of 1 day in one or multiple (e g., 2, 3, 4, or 5) administrations.

In another specific embodiment, a single dose of Alanyl-Glutamine as used in accordance with the methods provided herein comprises about 30-60 grams of Alanyl-Glutamine. In another specific embodiment, said dose of Alanyl-Glutamine as used in accordance with the methods is administered with zinc, e.g., 100-400 mg of zinc. In certain embodiments, said dose is administered over the course of 1 day in one or multiple (e.g., 2, 3, 4, or 5) administrations.

Doses of Alanyl-Glutamine relevant to the methods described herein are further described in Section 5.2.1.

The patients treated in accordance with the methods described herein can be diagnosed with any type of cancer. A non-limiting list of cancers for which treatment can cause dose-limiting side effects is presented in Section 5.2.4. In a specific embodiment, a patient administered a Alanyl-Glutamine formulation described herein in order to treat or prevent one or more cancer treatment side effects has been diagnosed with head and neck cancer. In another specific embodiment, a patient administered a Alanyl-Glutamine formulation described herein in order to treat or prevent one or more cancer treatment side effects has been diagnosed with breast cancer. In another specific embodiment, a patient administered a Alanyl-Glutamine formulation described herein in order to treat or prevent one or more cancer treatment side effects has been diagnosed with lung cancer. In another specific embodiment, a patient administered a Alanyl-Glutamine formulation described herein in order to treat or prevent one or more cancer treatment side effects has been diagnosed with myeloma.

The patients treated in accordance with the methods described herein can be experiencing side effects resulting from treatment with any type of cancer treatment. A non-limiting list of cancers for which treatment can cause dose-limiting side effects is presented in Section 5.2.4. In certain embodiments, a patient treated in accordance with the methods described herein has not been diagnosed with cancer, but is receiving a cancer treatment as a means to treat a disease other than cancer. Such patients/diseases/treatments are described in Section 5.2.3.

In another aspect, provided herein are sealed and/or capped beverage containers comprising an Alanyl-Glutamine formulation described herein. In a specific embodiment, provided herein is a sealed and/or capped bottle comprising an Alanyl-Glutamine formulation described herein, wherein said bottle is capable of holding a volume of 1, 2, 3, 4, 5, 6, 7, 8, 9, or 10 ounces. In another specific embodiment, the Alanyl-Glutamine formulation present in a sealed beverage container described herein comprises zinc. See Section 6.

### 3.1 Definitions

As used herein, the term “about,” when used in conjunction with a number, refers to any number within ±1, +5, or ±10% of the referenced number.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., an Alanyl-Glutamine formulation described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

As used herein, the term “patient” refers to a human that has received or is expected to receive a cancer treatment. The terms “subject” and “patient” may be used herein interchangeably herein.

As used herein, the term “Alanyl-Glutamine” refers to a glutamine dipeptide consisting of a glutamine amino group joined to an alanyl residue, and includes all related nomenclature, including the following: L-alanyl-L-glutamine; Alanyl-L-Glutamine; Glutamine-S, alanylglutamine; N(2)-L-alanyl-L-glutamine; N-L-alanyl-L-glutamine; Ala-Gln; AlaGln; AQ, L-alanine-glutamine; L-Ala-L-Gln; L-Ala-L-Gln-OH; L-alanyl-L-glutamine Ihydrate; L-alanyl-L-glutamine monohydrate; H-Ala-Gln-OH; C8H15N304; (2S)-2-[(2S)-2-aminopropanamido]-4-carbamoylbutanoic acid; (28)-2-((2S)-2-aminopropanoylamino)-4-carbamoylbutanoic acid; (2S)-5-amino-2-[(2S)-2-aminopropanoyl]amino]-5-oxo-pentanoic acid; (2S)-5-amino-2-[(2S)-2-aminopropanoyl]amino]-5-oxopentanoic acid).

As used herein, the terms “cancer treatment side effect” and “side effects associated with cancer treatment” mean any side effect or toxicity that impacts a patient receiving a cancer treatment (e.g., chemotherapy or radiation). Cancer treatment side effects can be side effects that are dose-limiting, i.e., result in a need to reduce and/or modify a dose of a cancer treatment a patient is receiving due to patient pain or discomfort. Cancer treatment side effects also can be side effects that are life threatening. Exemplary cancer treatment side effects include, without limitation, oral mucositis, peripheral neuropathy, cardiac damage (e.g., cardiomyopathy), dysgeusia, and hypertension.

As used herein, the terms “treat,” “treatment,” and “treating” in the context of the methods described herein refer to a beneficial or therapeutic effect resulting from administration of an Alanyl-Glutamine formulation described herein to a subject undergoing cancer treatment. In specific embodiments, such terms refer to one, two, three, four, five or more of the following effects: (i) reduction or amelioration in the severity of a cancer treatment side effect or a symptom associated therewith; (ii) reduction in the duration of a cancer treatment side effect or a symptom associated therewith; (iii) prevention of the progression of a cancer treatment side effect or a symptom associated therewith; (iv) regression of a cancer treatment side effect or a symptom associated therewith; (v) prevention of the development or onset of a cancer treatment side effect or a symptom associated therewith; (vi) prevention of the recurrence of a cancer treatment side effect or a symptom associated therewith; (vii) reduction in organ failure associated with a cancer treatment side effect; (viii) reduction in the hospitalization of a subject undergoing cancer treatment, (ix) reduction in the hospitalization length of a subject undergoing cancer treatment; (x) an increase in the survival of a subject undergoing cancer treatment; (xi) reduction in the number of symptoms and/or side effects associated with a cancer treatment; and/or (xii) enhancement, improvement, supplementation, complementation, or augmentation of the prophylactic or therapeutic effect(s) of another therapy.

As used herein, the terms “prevent,” “preventing” and “prevention” in the context of the methods described herein refers to a prophylactic effect resulting from administration of an Alanyl-Glutamine formulation described herein to a subject undergoing cancer treatment. In a specific embodiment, the terms “prevent,” “preventing” and “prevention” in the context of the refer to one or more of the following effects: (i) the inhibition or reduction in the development or onset of a cancer treatment side effect or a symptom thereof; (ii) the inhibition or reduction in the recurrence of a cancer treatment side effect or a symptom associated therewith; and/or (iii) prevention of the onset or development of one or more symptoms associated with a cancer treatment side effect.

As used herein the term “cancer treatment” refers to any therapy used or contemplated for use in treatment of a cancer. Exemplary cancer treatments include chemotherapy and radiation.

### 4. BRIEF DESCRIPTION OF THE DRAWINGS

FIG. 1 depicts number of surviving intestinal crypt cells of irradiated mice treated with control (“vehicle”), Alanyl-Glutamine (“Ala-Gln”), Glutamine (“Gln”), or Alanyl-Glutamine and zinc (“Ala-Gln+Zn”). Bars depict results when mice were pre-treated or when treatment was initiated on the day of radiation.

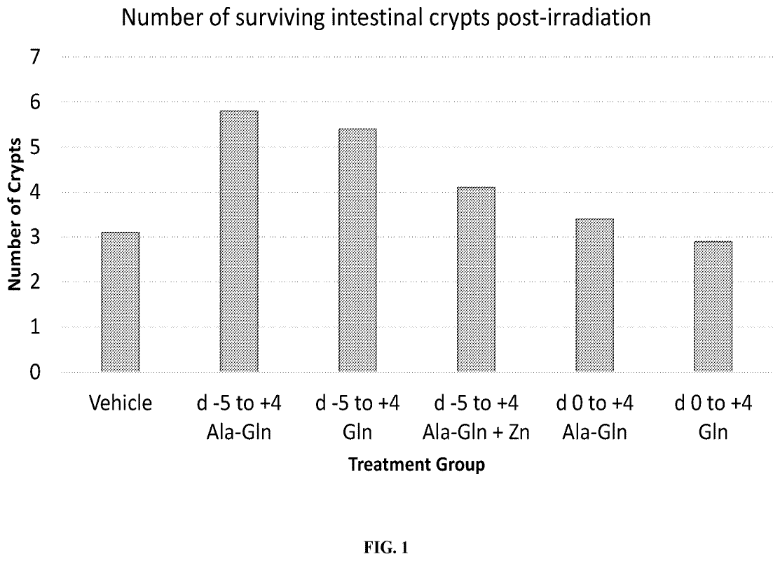

FIGS. 2A-C depicts graphs demonstrating an increase in glutamine in circulating plasma following oral administration of Alanyl-Glutamine (Ala-Gln) and glutamine (Gln) (i.e., a glutamine amino group that is not joined to an alanyl residue) in rats. Either Alanyl-Glutamine or glutamine was administered as a single oral dose at three different dose levels, low (2A), medium and high, with the doses at each level containing molar equivalent amounts of Glutamine. Following administration, plasma glutamine levels were assessed at 9 time-points, and the resulting changes over baseline glutamine levels were averaged and graphed for each time-point. Plasma glutamine was increased more following oral Alanyl-Glutamine than oral glutamine administration, despite each dose containing the same glutamine concentration overall. This result was observed at all three dose levels, and indicates that oral Alanyl-Glutamine is more efficient at increasing plasma glutamine than glutamine in vivo.

### 5. DETAILED DESCRIPTION

The present invention generally relates to preventing, managing, ameliorating or treating oral mucositis and/or peripheral neuropathy and/or dysgeusia and/or cardiac damage (e.g., cardiomyopathy) by administering to a patient in need thereof, a low volume beverage containing alanyl-glutamine or a combination product comprising alanyl-glutamine and zinc as a prophylactic regimen or therapeutically effective regimen, the regimen comprising administration to the patient orally daily with prior to and/or concurrent with and/or following cancer therapy. Reduction in the incidence, severity and/or duration of oral mucositis, peripheral neuropathy, dysgeusia, and/or cardiac damage (e.g., cardiomyopathy), which represent major dose-limiting toxicities associated with chemotherapy and radiation therapy, would result in an increased number of patients that can tolerate the full treatment cycle and treatment dose Successful completion of the full course of chemotherapy or radiation therapy allows patients to achieve maximal effectiveness, tumor response, progression free survival, reduction of metastasis, reduction in the rate of relapse, increased duration of remission, higher remission rate, longer duration of treatment, increased intensity of chemotherapy and radiation, and increased overall survival.

### 5.1 Formulations

Provided herein are Alanyl-Glutamine formulations for use in treating and preventing side effects associated with cancer treatment, e.g., side effects caused by chemotherapy and/or radiation. The Alanyl-Glutamine formulations provided herein can be formulated for oral administration, e.g., as a beverage, and can comprise one or more additional components. In a specific embodiment, said Alanyl-Glutamine formulations further comprise zinc.

In a specific embodiment, the Alanyl-Glutamine formulations provided herein comprise Alanyl-Glutamine in an appropriate solution (e.g., water) Such formulations can, in certain embodiments, comprise about 1 gram of Alanyl-Glutamine/ounce of solution, about 5 grams of Alanyl-Glutamine/ounce of solution, about 10 grams of Alanyl-Glutamine/ounce of solution, about 15 grams of Alanyl-Glutamine/ounce of solution, about 20 grams of Alanyl-Glutamine/ounce of solution, about 25 grams of Alanyl-Glutamine/ounce of solution, about 30 grams of Alanyl-Glutamine/ounce of solution, about 35 grams of Alanyl-Glutamine/ounce of solution, about 40 grams of Alanyl-Glutamine/ounce of solution, about 45 grams of Alanyl-Glutamine/ounce of solution, about 50 grams of Alanyl-Glutamine/ounce of solution, about 55 grams of Alanyl-Glutamine/ounce of solution, about 60 grams of Alanyl-Glutamine/ounce of solution, about 65 grams of Alanyl-Glutamine/ounce of solution, about 70 grams of Alanyl-Glutamine/ounce of solution, about 75 grams of Alanyl-Glutamine/ounce of solution, about 80 grams of Alanyl-Glutamine/ounce of solution, about 85 grams of Alanyl-Glutamine/ounce of solution, about 90 grams of Alanyl-Glutamine/ounce of solution, about 95 grams of Alanyl-Glutamine/ounce of solution, or about 100 grams of Alanyl-Glutamine/ounce of solution.

In a specific embodiment, the Alanyl-Glutamine formulations provided herein comprise about 1-5 grams of Alanyl-Glutamine/ounce of solution, about 5-10 grams of Alanyl-Glutamine/ounce of solution, about 10-15 grams of Alanyl-Glutamine/ounce of solution, about 15-20 grams of Alanyl-Glutamine/ounce of solution, about 20-25 grams of Alanyl-Glutamine/ounce of solution, about 25-30 grams of Alanyl-Glutamine/ounce of solution, about 30-35 grams of Alanyl-Glutamine/ounce of solution, about 35-40 grams of Alanyl-Glutamine/ounce of solution, about 40-45 grams of Alanyl-Glutamine/ounce of solution, about 45-50 grams of Alanyl-Glutamine/ounce of solution, about 50-55 grams of Alanyl-Glutamine/ounce of solution, about 55-60 grams of Alanyl-Glutamine/ounce of solution, about 60-65 grams of Alanyl-Glutamine/ounce of solution, about 65-70 grams of Alanyl-Glutamine/ounce of solution, about 70-75 grams of Alanyl-Glutamine/ounce of solution, about 75-80 grams of Alanyl-Glutamine/ounce of solution, about 80-85 grams of Alanyl-Glutamine/ounce of solution, about 85-90 grams of Alanyl-Glutamine/ounce of solution, about 90-95 grams of Alanyl-Glutamine/ounce of solution, or about 95-100 grams of Alanyl-Glutamine/ounce of solution.

In another specific embodiment, an Alanyl-Glutamine formulation provided herein comprises about 1-100 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 5-60 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 10-30 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 20-30 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 10 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 14.8 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 15 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 19.7 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 20 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 22.2 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 30 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 44.4 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In another specific embodiment, an Alanyl-Glutamine formulation provided herein comprises about 59.2 grams of Alanyl-Glutamine in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In certain embodiments, the Alanyl-Glutamine formulations provided herein, including any of the foregoing formulations, comprise zinc (e.g., zinc from zinc sulfate monohydrate or heptahydrate). In a specific embodiment, said formulations comprise about 1 mg zinc/ounce of solution, about 5 mg zinc/ounce of solution, about 10 mg zinc/ounce of solution, about 15 mg zinc/ounce of solution, about 20 mg zinc/ounce of solution, about 25 mg zinc/ounce of solution, about 30 mg zinc/ounce of solution, about 35 mg zinc/ounce of solution, about 40 mg zinc/ounce of solution, about 45 mg zinc/ounce of solution, about 50 mg zinc/ounce of solution, about 55 mg zinc/ounce of solution, about 60 mg zinc/ounce of solution, about 65 mg zinc/ounce of solution, about 70 mg zinc/ounce of solution, about 75 mg zinc/ounce of solution, about 80 mg zinc/ounce of solution, about 85 mg zinc/ounce of solution, about 90 mg zinc/ounce of solution, about 95 mg zinc/ounce of solution, about 100 mg zinc/ounce of solution, about 125 mg zinc/ounce of solution, about 150 mg zinc/ounce of solution, about 175 mg zinc/ounce of solution, about 200 mg zinc/ounce of solution, about 250 mg zinc/ounce of solution, about 300 mg zinc/ounce of solution, about 400 mg zinc/ounce of solution, or about 500 mg zinc/ounce of solution.

In certain embodiments, the Alanyl-Glutamine formulations provided herein, including any of the foregoing formulations, comprise about 1-5 mg zinc/ounce of solution, about 5-10 mg zinc/ounce of solution, about 10-15 mg zinc/ounce of solution, about 15-20 mg zinc/ounce of solution, about 20-25 mg zinc/ounce of solution, about 25-30 mg zinc/ounce of solution, about 30-35 mg zinc/ounce of solution, about 35-40 mg zinc/ounce of solution, about 40-45 mg zinc/ounce of solution, about 45-50 mg zinc/ounce of solution, about 50-55 mg zinc/ounce of solution, about 55-60 mg zinc/ounce of solution, about 60-65 mg zinc/ounce of solution, about 65-70 mg zinc/ounce of solution, about 70-75 mg zinc/ounce of solution, about 75-80 mg zinc/ounce of solution, about 80-85 mg zinc/ounce of solution, about 85-90 mg zinc/ounce of solution, about 90-95 mg zinc/ounce of solution, about 95-100 mg zinc/ounce of solution, about 100-125 mg zinc/ounce of solution, about 125-150 mg zinc/ounce of solution, about 150-175 mg zinc/ounce of solution, about 175-200 mg zinc/ounce of solution, about 200-250 mg zinc/ounce of solution, about 250-300 mg zinc/ounce of solution, about 300-400 mg zinc/ounce of solution, or about 400-500 mg zinc/ounce of solution.

In a specific embodiment, an Alanyl-Glutamine formulation provided herein comprises about 1-100 grams of Alanyl-Glutamine and about 10-200 mg of zinc in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In a specific embodiment, an Alanyl-Glutamine formulation provided herein comprises about 5-60 grams of Alanyl-Glutamine and about 10-100 mg of zinc in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In a specific embodiment, an Alanyl-Glutamine formulation provided herein comprises about 10-30 grams of Alanyl-Glutamine and about 10-50 mg of zinc in solution (e.g., in water), wherein the total volume of the solution can range from about 0.1 ounces to about 1 liter. In a specific embodiment, the total volume of the solution is about 0.5 ounces. In another specific embodiment, the total volume of the solution is about 1 ounce. In another specific embodiment, the total volume of the solution is about 1.5 ounces. In another specific embodiment, the total volume of the solution is about 2 ounces. In another specific embodiment, the total volume of the solution is about 2.5 ounces. In another specific embodiment, the total volume of the solution is about 3 ounces. In another specific embodiment, the total volume of the solution is about 3.5 ounces. In another specific embodiment, the total volume of the solution is about 4 ounces. In another specific embodiment, the total volume of the solution is about 4.5 ounces. In another specific embodiment, the total volume of the solution is about 5 ounces.

In certain embodiments, the Alanyl-Glutamine formulations provided herein can comprise one or more of the following additional components: sweeteners (e.g., glucose, maltodextrin, maltose, dextrose, high fructose corn syrup, sucrose, lactose, fructose, Saccharin, aspartame, acesulfame K, sucralose, neotame, rebaudioside A, Stevia/Erythritol, Sorbitol, Mannitol, and Xylitol), thickening and/or gelling agents (e.g., alginate, propylene glycol alginate, carrageenans, cellulose gum and microcrystalline cellulose, Xanthum gums, and guar gums), coloring agents (e.g., Blue No. 2 (Indigotine), Red No. 40 (Allura Red AC), Yellow No. 5 (Tartrazine)); flavor and fragrance agents; agents capable of maintaining the formulations at a desired pH (e.g., citric acid, sodium citrate, zinc sulphase, magnesium sulphate, potassium chloride, and magnesium oxide); vitamins (e.g., vitamins A, D, E, K, B, C, Niacin, and folic acids); masking agents (e.g., citric acid, sodium citrates, sodium phosphate, and hydroxyl acids); and/or bitter taste-receptor blockers (e.g, hydrogenated ethoxylated glycerol ester, Beta-Cyclodextrin, homoeriodictyol sodium salt, and eriodictyol). In one embodiment, the beverage may contain from 0.1% to 10% sweeteners and all percentages to the tenth decimal place there between. The sweeteners may be nutritive and non-nutritive, natural and artificial or synthetic.

In certain embodiments the formulations provided herein comprise one or more additives and/or excipients that improve the bioavailabity of Alanyl-Glutamine. In certain embodiments the formulations provided herein comprise one or more additives and/or excipients that improve the stability of Alanyl-Glutamine.

In certain embodiments, the Alanyl-Glutamine formulations provided herein are present in a container (e.g., a bottle, flask, tube, jar) that can be provided to a subject in need of ingestion of an Alanyl-Glutamine formulation, e.g., a patient undergoing cancer treatment or that is scheduled to undergo a cancer treatment. In a specific embodiment, a single container comprising an Alanyl-Glutamine formulation comprises a single dose of Alanyl-Glutamine. In another specific embodiment, a single container comprising an Alanyl-Glutamine formulation comprises multiple doses of Alanyl-Glutamine, e.g., 1, 2, 3, 4, 5, 6, 7, 8, 9, or 10 doses of Alanyl-Glutamine. In another specific embodiment, a single container comprising an Alanyl-Glutamine formulation comprises a portion of a single dose of Alanyl-Glutamine, e.g., one-fifth, one-quarter, one-third, or one-half of a dose of Alanyl-Glutamine.

In certain embodiments, the Alanyl-Glutamine formulations provided herein have a calorie content of less than 1. In a specific embodiment, the calorie content is 0. In another specific embodiment, the calorie content is less than 5. In another specific embodiment the caloric content is between 1 and 50 calories. In certain embodiments, the Alanyl-Glutamine formulations provided herein have more than 50 calories.

In certain embodiments, the Alanyl-Glutamine formulations provided herein can be prepared from a powdered mix. Accordingly, also provided herein are powdered compositions that, when suspended in appropriate solution (e.g, water), forms an Alanyl-Glutamine formulation provided herein.

In certain embodiments, the Alanyl-Glutamine formulations provided herein are in the form of a gel or a goo.

In certain embodiments, the Alanyl-Glutamine formulations provided herein are provided as a frozen ice.

In certain embodiments, the Alanyl-Glutamine formulations provided herein are in provided as a concentrated liquid that, when diluted in appropriate solution (e.g., water), forms an Alanyl-Glutamine formulation provided herein.

### 5.2 Methods

In another aspect, provided herein are methods of treating or preventing side effects associated with cancer treatment, wherein said methods comprise oral administration of an Alanyl-Glutamine formulation described herein. In a specific embodiment, the Alanyl-Glutamine formulation comprises zinc. In another specific embodiment, the Alanyl-Glutamine formulation is administered as a beverage (e.g., a liquid formulation that the subject can drink).

In a specific embodiment, the methods of treating or preventing side effects associated with cancer treatment provided herein treat or prevent side effects caused by administration of chemotherapy.

In another specific embodiment, the methods of treating or preventing side effects associated with cancer treatment provided herein treat or prevent side effects caused by administration of radiation.

In another specific embodiment, the side effect caused by cancer treatment that is treated or prevented by the methods provided herein is oral mucositis. See Section 5.2.3.1.

In another specific embodiment, the side effect caused by cancer treatment that is treated or prevented by the methods provided herein is peripheral neuropathy See Section 5.2.3.2.

In another specific embodiment, the side effect caused by cancer treatment that is treated or prevented by the methods provided herein is cardiac damage (e.g., cardiomyopathy). See Section 5.2.3.3.

In another specific embodiment, the side effect caused by cancer treatment that is treated or prevented by the methods provided herein is dysgeusia. See Section 5.2.3.4.

In another specific embodiment, the side effect caused by cancer treatment that is treated or prevented by the methods provided herein is hypertension. See Section 5.2.3.5.

In certain embodiments of the methods of treating or preventing side effects associated with cancer treatment provided herein, a patient diagnosed with cancer is administered an Alanyl-Glutamine formulation described herein prior to initiation of a cancer treatment regimen, e.g., before administration of chemotherapy and/or radiation. In a specific embodiment, the patient is administered an Alanyl-Glutamine formulation described herein 1, 2, 3, or 4 weeks prior to initiation of a cancer treatment regimen. In another specific embodiment, the patient is administered an Alanyl-Glutamine formulation described herein 1, 2, 3, 4, 5, 6, 7, 8, 9, 10, 11, 12, 13, 14, 15, 16, 17, 18, 19, 20, or 21 days prior to initiation of a cancer treatment regimen. In another specific embodiment, the patient is administered an Alanyl-Glutamine formulation described herein on the same day that a cancer treatment regimen begins, but before a cancer treatment is administered to the patient. In another specific embodiment, once a patient has first been administered an Alanyl-Glutamine formulation described herein (day 1), they are administered an Alanyl-Glutamine formulation described herein daily for the course of the cancer treatment. In a specific embodiment, the side effect caused by cancer treatment that is treated or prevented is oral mucositis, peripheral neuropathy, cardiac damage (e.g., cardiomyopathy), dysgeusia, and/or hypertension.

In certain embodiments of the methods of treating or preventing side effects associated with cancer treatment provided herein, a patient diagnosed with cancer is administered an Alanyl-Glutamine formulation described herein after initiation of a cancer treatment regimen, e.g., after administration of chemotherapy and/or radiation. In a specific embodiment, the patient is administered an Alanyl-Glutamine formulation described herein 1, 2, 3, or 4 weeks after initiation of a cancer treatment regimen. In another specific embodiment, the patient is administered an Alanyl-Glutamine formulation described herein 1, 2, 3, 4, 5, 6, 7, 8, 9, 10, 11, 12, 13, 14, 15, 16, 17, 18, 19, 20, or 21 days after initiation of a cancer treatment regimen. In another specific embodiment, the patient is administered an Alanyl-Glutamine formulation described herein on the same day that a cancer treatment regimen begins, but after a cancer treatment is administered to the patient. In another specific embodiment, once a patient has first been administered an Alanyl-Glutamine formulation described herein (day 1), they are administered an Alanyl-Glutamine formulation described herein daily for the course of the cancer treatment. In a specific embodiment, the side effect caused by cancer treatment that is treated or prevented is oral mucositis, peripheral neuropathy, cardiac damage (e.g., cardiomyopathy), dysgeusia, and/or hypertension.

In certain embodiments of the methods of treating or preventing side effects associated with cancer treatment provided herein, a patient diagnosed with cancer is administered an Alanyl-Glutamine formulation described herein for the duration of the cancer treatment, e.g., as long as the patient is being treated for cancer (e.g., being administered chemotherapy or radiation, or both), the patient is administered an Alanyl-Glutamine formulation described herein. In a specific embodiment, the patient is administered an Alanyl-Glutamine formulation described herein daily for the course of the cancer treatment. In a specific embodiment, the side effect caused by cancer treatment that is treated or prevented is oral mucositis, peripheral neuropathy, cardiac damage (e.g., cardiomyopathy), dysgeusia, and/or hypertension.

In certain embodiments of the methods of treating or preventing side effects associated with cancer treatment provided herein, a patient diagnosed with cancer is administered an Alanyl-Glutamine formulation described herein after a cancer treatment has been completed, e.g., the patient is administered an Alanyl-Glutamine formulation described herein for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after the cancer treatment has been completed (e.g., after the final dose of chemotherapy and/or radiation); for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks after the cancer treatment has been completed (e.g., after the final dose of chemotherapy and/or radiation); or for 1, 2, 3, 4, or 5 years after the cancer treatment has been completed (e.g., after the final dose of chemotherapy and/or radiation). In a specific embodiment, the patient is administered an Alanyl-Glutamine formulation described herein daily after the cancer treatment has been completed. In a specific embodiment, the side effect caused by cancer treatment that is treated or prevented is oral mucositis, peripheral neuropathy, cardiac damage (e.g., cardiomyopathy), dysgeusia, and/or hypertension.

In a specific embodiment of the methods of treating or preventing side effects associated with cancer treatment provided herein, a patient diagnosed with cancer is administered an Alanyl-Glutamine formulation described herein (i) prior to initiation of a cancer treatment regimen (e.g., before administration of chemotherapy and/or radiation), for example, 1, 2, 3, 4, 5, 6, 7, 8, 9, 10, 11, 12, 13, 14, 15, 16, 17, 18, 19, 20, or 21 days prior to initiation of a cancer treatment regimen; or 1, 2, 3, or 4 weeks prior to initiation of a cancer treatment regimen; and (ii) for the duration of the cancer treatment, e.g., as long as the patient is being treated for cancer (e.g., being administered chemotherapy or radiation, or both). In a specific embodiment, the side effect caused by cancer treatment that is treated or prevented is oral mucositis, peripheral neuropathy, cardiac damage (e.g., cardiomyopathy), dysgeusia, and/or hypertension.

In another specific embodiment of the methods of treating or preventing side effects associated with cancer treatment provided herein, a patient diagnosed with cancer is administered an Alanyl-Glutamine formulation described herein (i) prior to initiation of a cancer treatment regimen (e.g., before administration of chemotherapy and/or radiation), for example, 1, 2, 3, 4, 5, 6, 7, 8, 9, 10, 11, 12, 13, 14, 15, 16, 17, 18, 19, 20, or 21 days prior to initiation of a cancer treatment regimen; or 1, 2, 3, or 4 weeks prior to initiation of a cancer treatment regimen; (ii) for the duration of the cancer treatment, e.g., as long as the patient is being treated for cancer (e.g., being administered chemotherapy or radiation, or both); and (iii) after the cancer treatment has been completed (e.g., after the final dose of chemotherapy and/or radiation), e.g., for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after the cancer treatment has been completed; for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks after the cancer treatment has been completed; or for 1, 2, 3, 4, or 5 years after the cancer treatment has been completed. In a specific embodiment, the side effect caused by cancer treatment that is treated or prevented is oral mucositis, peripheral neuropathy, cardiac damage (e g., cardiomyopathy), dysgeusia, and/or hypertension.

In accordance with the methods of treating or preventing side effects associated with cancer treatment provided herein, it is to be understood that administration of an Alanyl-Glutamine formulation described herein to a patient in need thereof, e.g., a subject undergoing or about to undergo a cancer treatment (e.g., chemotherapy or radiation) is performed in a manner that results in the patient receiving a dose of Alanyl-Glutamine. A dose of Alanyl-Glutamine can be administered as part of a single administration or as part of multiple administrations, wherein said multiple administrations can occur over the course of 1, 2, 3, 4, 5, 6, or 7 days. In a specific embodiment, said multiple administrations occur over the course of 1 day.

In certain embodiments, a dose of Alanyl-Glutamine is administered over the course of 1 day on a daily basis as part of a method described herein, wherein the daily dose is divided over the course of 2, 3, 4, 5, 6, 7, 8, 9, or 10 administrations of an Alanyl-Glutamine formulation described herein. In a specific embodiment, a dose of Alanyl-Glutamine is administered on a daily basis as part of a method described herein, wherein the daily dose is divided over the course of 2 administrations of an Alanyl-Glutamine formulation described herein, i.e., a patient is administered an Alanyl-Glutamine formulation described herein twice a day. In another specific embodiment, a dose of Alanyl-Glutamine is administered on a daily basis as part of a method described herein, wherein the daily dose is divided over the course of 3 administrations of an Alanyl-Glutamine formulation described herein, i.e., a patient is administered an Alanyl-Glutamine formulation described herein three times a day. In another specific embodiment, a dose of Alanyl-Glutamine is administered on a daily basis as part of a method described herein, wherein the daily dose is divided over the course of 4 administrations of an Alanyl-Glutamine formulation described herein, i.e., a patient is administered an Alanyl-Glutamine formulation described herein four times a day. In another specific embodiment, a dose of Alanyl-Glutamine is administered on a daily basis as part of a method described herein, wherein the daily dose is divided over the course of 5 administrations of an Alanyl-Glutamine formulation described herein, i.e., a patient is administered an Alanyl-Glutamine formulation described herein five times a day.

In certain embodiments, a dose of Alanyl-Glutamine is administered on a daily basis as part of a method described herein, wherein the daily dose is not divided over the course of multiple administrations, but instead is administered as part of a single administration, i.e., a patient is administered an Alanyl-Glutamine formulation described herein once a day.

In certain embodiments, a single dose of Alanyl-Glutamine as used in accordance with the methods provided herein comprises about 5 grams of Alanyl-Glutamine, about 10 grams of Alanyl-Glutamine, about 15 grams of Alanyl-Glutamine, about 20 grams of Alanyl-Glutamine/ounce of solution, about 25 grams of Alanyl-Glutamine, about 30 grams of Alanyl-Glutamine, about 35 grams of Alanyl-Glutamine, about 40 grams of Alanyl-Glutamine, about 45 grams of Alanyl-Glutamine, about 50 grams of Alanyl-Glutamine, about 55 grams of Alanyl-Glutamine, about 60 grams of Alanyl-Glutamine, about 65 grams of Alanyl-Glutamine, about 70 grams of Alanyl-Glutamine, about 75 grams of Alanyl-Glutamine, about 80 grams of Alanyl-Glutamine, about 85 grams of Alanyl-Glutamine, about 90 grams of Alanyl-Glutamine, about 95 grams of Alanyl-Glutamine, or about 100 grams of Alanyl-Glutamine. In a specific embodiment, said single dose of Alanyl-Glutamine is administered with zinc (e.g., a solution that comprises both Alanyl-Glutamine and zinc is administered or a solution that comprises Alanyl-Glutamine and a separate solution that comprises zinc are administered), wherein about 10, 15, 20, 25, 30, 35, 40, 45, 50, 55, 60, 65, 70, 75, 80, 85, 90, 95, 100, 125, 150, 175, 200, 225, 250, 300, 350, 400, 450, or 500 mg of zinc is administered with said dose of Alanyl-Glutamine. In another specific embodiment, said single dose of Alanyl-Glutamine is administered with zinc (e.g., a solution that comprises both Alanyl-Glutamine and zinc is administered or a solution that comprises Alanyl-Glutamine and a separate solution that comprises zinc are administered), wherein about 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-125, 125-150, 150-175, 175-200, 200-250, 250-300, 300-350, 350-400, 400-450, or 450-500 mg of zinc is administered with said dose of Alanyl-Glutamine. In certain embodiments, said dose of Alanyl-Glutamine or said dose of Alanyl-Glutamine and zinc is administered over the course of 1 day in one or multiple (e.g., 2, 3, 4, or 5) administrations.

In certain embodiments, a single dose of Alanyl-Glutamine as used in accordance with the methods provided herein comprises about 5-10 grams of Alanyl-Glutamine, about 10-15 grams of Alanyl-Glutamine, about 15-20 grams of Alanyl-Glutamine, about 20-25 grams of Alanyl-Glutamine, about 25-30 grams of Alanyl-Glutamine, about 30-35 grams of Alanyl-Glutamine, about 35-40 grams of Alanyl-Glutamine about 40-45 grams of Alanyl-Glutamine, about 45-50 grams of Alanyl-Glutamine, about 50-55 grams of Alanyl-Glutamine, about 55-60 grams of Alanyl-Glutamine, about 60-65 grams of Alanyl-Glutamine, about 65-70 grams of Alanyl-Glutamine, about 70-75 grams of Alanyl-Glutamine, about 75-80 grams of Alanyl-Glutamine, about 80-85 grams of Alanyl-Glutamine, about 85-90 grams of Alanyl-Glutamine, about 90-95 grams of Alanyl-Glutamine, or about 95-100 grams of Alanyl-Glutamine. In a specific embodiment, said single dose of Alanyl-Glutamine is administered with zinc (e.g., a solution that comprises both Alanyl-Glutamine and zinc is administered or a solution that comprises Alanyl-Glutamine and a separate solution that comprises zinc are administered), wherein about 10, 15, 20, 25, 30, 35, 40, 45, 50, 55, 60, 65, 70, 75, 80, 85, 90, 95, 100, 125, 150, 175, 200, 225, or 250, 300, 350, 400, 450, or 500 mg of zinc is administered with said dose of Alanyl-Glutamine. In another specific embodiment, said single dose of Alanyl-Glutamine is administered with zinc (e.g., a solution that comprises both Alanyl-Glutamine and zinc is administered or a solution that comprises Alanyl-Glutamine and a separate solution that comprises zinc are administered), wherein about 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-125, 125-150, 150-175, 175-200, 200-250, 250-300, 300-350, 350-400, 400-450, or 450-500 mg of zinc is administered with said dose of Alanyl-Glutamine. In certain embodiments, said dose of Alanyl-Glutamine or said dose of Alanyl-Glutamine and zinc is administered over the course of 1 day in one or multiple (e.g., 2, 3, 4, or 5) administrations.

In a specific embodiment, a single dose of Alanyl-Glutamine as used in accordance with the methods provided herein comprises about 20-75 grams of Alanyl-Glutamine. In another specific embodiment, said dose of Alanyl-Glutamine as used in accordance with the methods is administered with zinc, e.g., 100-400 mg of zinc. In certain embodiments, said dose is administered over the course of 1 day in one or multiple (e.g., 2, 3, 4, or 5) administrations.

In another specific embodiment, a single dose of Alanyl-Glutamine as used in accordance with the methods provided herein comprises about 20-60 grams of Alanyl-Glutamine. In another specific embodiment, said dose of Alanyl-Glutamine as used in accordance with the methods is administered with zinc, e.g., 100-400 mg of zinc. In certain embodiments, said dose is administered over the course of 1 day in one or multiple (e.g., 2, 3, 4, or 5) administrations.

In another specific embodiment, a single dose of Alanyl-Glutamine as used in accordance with the methods provided herein comprises about 30-60 grams of Alanyl-Glutamine. In another specific embodiment, said dose of Alanyl-Glutamine as used in accordance with the methods is administered with zinc, e.g., 100-400 mg of zinc. In certain embodiments, said dose is administered over the course of 1 day in one or multiple (e.g., 2, 3, 4, or 5) administrations.

Doses of Alanyl-Glutamine relevant to the methods described herein are further described in Section 5.2.1.

The patients treated in accordance with the methods described herein can be diagnosed with any type of cancer. A non-limiting list of cancers for which treatment can cause dose-limiting side effects is presented in Section 5.2.4. In a specific embodiment, a patient administered a Alanyl-Glutamine formulation described herein in order to treat or prevent one or more cancer treatment side effects has been diagnosed with head and neck cancer. In another specific embodiment, a patient administered a Alanyl-Glutamine formulation described herein in order to treat or prevent one or more cancer treatment side effects has been diagnosed with breast cancer. In another specific embodiment, a patient administered a Alanyl-Glutamine formulation described herein in order to treat or prevent one or more cancer treatment side effects has been diagnosed with lung cancer. In another specific embodiment, a patient administered a Alanyl-Glutamine formulation described herein in order to treat or prevent one or more cancer treatment side effects has been diagnosed with myeloma.

The patients treated in accordance with the methods described herein can be experiencing side effects resulting from treatment with any type of cancer treatment. A non-limiting list of cancers for which treatment can cause dose-limiting side effects is presented in Section 5.2.4. In certain embodiments, a patient treated in accordance with the methods described herein has not been diagnosed with cancer, but is receiving a cancer treatment as a means to treat a disease other than cancer. Such patients/diseases/treatments are described in Section 5.2.3.

### 5.2.1 Doses

As used herein, the term “dose,” when referring to a dose of Alanyl-Glutamine, refers to an amount of Alanyl-Glutamine to be administered as a part of a treatment regimen that includes administration of multiple doses of Alanyl-Glutamine over a period of time (e.g., a defined period of time, such as one week, one month, or one year; or a period of time that is indefinite, e.g., over the course of time that a subject requires treatment for cancer). A dose of Alanyl-Glutamine can be administered in one day, or over the course of multiple days. In addition, a dose of Alanyl-Glutamine can be administered as part of a single formulation (e.g., in one administration) or administration of a dose of Alanyl-Glutamine can comprise multiple administrations of an amount of Alanyl-Glutamine with a specified number of administrations comprising a single dose. In a specific embodiment, a dose of Alanyl-Glutamine is administered over the course of one day (e.g., via one or multiple administrations of a an Alanyl-Glutamine formulation described herein).

In certain embodiments, a single dose of Alanyl-Glutamine (e.g., a total daily dose of Alanyl-Glutamine) comprises about 5 grams of Alanyl-Glutamine, about 10 grams of Alanyl-Glutamine, about 15 grams of Alanyl-Glutamine, about 20 grams of Alanyl-Glutamine/ounce of solution, about 25 grams of Alanyl-Glutamine, about 30 grams of Alanyl-Glutamine, about 35 grams of Alanyl-Glutamine, about 40 grams of Alanyl-Glutamine, about 45 grams of Alanyl-Glutamine, about 50 grams of Alanyl-Glutamine, about 55 grams of Alanyl-Glutamine, about 60 grams of Alanyl-Glutamine, about 65 grams of Alanyl-Glutamine, about 70 grams of Alanyl-Glutamine, about 75 grams of Alanyl-Glutamine, about 80 grams of Alanyl-Glutamine, about 85 grams of Alanyl-Glutamine, about 90 grams of Alanyl-Glutamine, about 95 grams of Alanyl-Glutamine, or about 100 grams of Alanyl-Glutamine. In a specific embodiment, said single dose of Alanyl-Glutamine (e.g., a total daily dose of Alanyl-Glutamine) is administered with zinc (e.g., a solution that comprises both Alanyl-Glutamine and zinc is administered or a solution that comprises Alanyl-Glutamine and a separate solution that comprises zinc are administered), wherein about 10, 15, 20, 25, 30, 35, 40, 45, 50, 55, 60, 65, 70, 75, 80, 85, 90, 95, 100, 125, 150, 175, 200, 225, 250, 300, 350, 400, 450, or 500 mg of zinc is administered with said dose of Alanyl-Glutamine. In another specific embodiment, said single dose of Alanyl-Glutamine is administered with zinc (e.g., a solution that comprises both Alanyl-Glutamine and zinc is administered or a solution that comprises Alanyl-Glutamine and a separate solution that comprises zinc are administered), wherein about 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-125, 125-150, 150-175, 175-200, 200-250, 250-300, 300-350, 350-400, 400-450, or 450-500 mg of zinc is administered with said dose of Alanyl-Glutamine.

In certain embodiments, a single dose of Alanyl-Glutamine (e.g., a total daily dose of Alanyl-Glutamine) comprises about 5-10 grams of Alanyl-Glutamine, about 10-15 grams of Alanyl-Glutamine, about 15-20 grams of Alanyl-Glutamine, about 20-25 grams of Alanyl-Glutamine, about 25-30 grams of Alanyl-Glutamine, about 30-35 grams of Alanyl-Glutamine, about 35-40 grams of Alanyl-Glutamine about 40-45 grams of Alanyl-Glutamine, about 45-50 grams of Alanyl-Glutamine, about 50-55 grams of Alanyl-Glutamine, about 55-60 grams of Alanyl-Glutamine, about 60-65 grams of Alanyl-Glutamine, about 65-70 grams of Alanyl-Glutamine, about 70-75 grams of Alanyl-Glutamine, about 75-80 grams of Alanyl-Glutamine, about 80-85 grams of Alanyl-Glutamine, about 85-90 grams of Alanyl-Glutamine, about 90-95 grams of Alanyl-Glutamine, or about 95-100 grams of Alanyl-Glutamine. In a specific embodiment, said single dose of Alanyl-Glutamine (e.g., a total daily dose of Alanyl-Glutamine) is administered with zinc (e.g., a solution that comprises both Alanyl-Glutamine and zinc is administered or a solution that comprises Alanyl-Glutamine and a separate solution that comprises zinc are administered), wherein about 10, 15, 20, 25, 30, 35, 40, 45, 50, 55, 60, 65, 70, 75, 80, 85, 90, 95, 100, 125, 150, 175, 200, 225, or 250, 300, 350, 400, 450, or 500 mg of zinc is administered with said dose of Alanyl-Glutamine. In another specific embodiment, said single dose of Alanyl-Glutamine is administered with zinc (e.g., a solution that comprises both Alanyl-Glutamine and zinc is administered or a solution that comprises Alanyl-Glutamine and a separate solution that comprises zinc are administered), wherein about 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-125, 125-150, 150-175, 175-200, 200-250, 250-300, 300-350, 350-400, 400-450, or 450-500 mg of zinc is administered with said dose of Alanyl-Glutamine.

In a specific embodiment, a single dose of Alanyl-Glutamine (e g., a total daily dose of Alanyl-Glutamine) comprises about 20-75 grams of Alanyl-Glutamine. In another specific embodiment, said dose of Alanyl-Glutamine is administered with zinc, e.g., 100-400 mg of zinc.

In another specific embodiment, a single dose of Alanyl-Glutamine (e.g., a total daily dose of Alanyl-Glutamine) comprises about 20-60 grams of Alanyl-Glutamine. In another specific embodiment, said dose of Alanyl-Glutamine is administered with zinc, e.g., 100-400 mg of zinc.

In another specific embodiment, a single dose of Alanyl-Glutamine (e.g., a total daily dose of Alanyl-Glutamine) comprises about 30-60 grams of Alanyl-Glutamine. In another specific embodiment, said dose of Alanyl-Glutamine as used in accordance with the methods is administered with zinc, e.g., 100-400 mg of zinc.

In another specific embodiment, a single dose of Alanyl-Glutamine (e.g., a total daily dose of Alanyl-Glutamine) comprises about 40-60 grams of Alanyl-Glutamine. In another specific embodiment, said dose of Alanyl-Glutamine as used in accordance with the methods is administered with zinc, e.g., 100-400 mg of zinc.

In another specific embodiment, a single dose of Alanyl-Glutamine (e.g., a total daily dose of Alanyl-Glutamine) comprises about 50-60 grams of Alanyl-Glutamine. In another specific embodiment, said dose of Alanyl-Glutamine as used in accordance with the methods is administered with zinc, e.g., 100-400 mg of zinc.

In another specific embodiment, a single dose of Alanyl-Glutamine (e.g., a total daily dose of Alanyl-Glutamine) comprises about 30 grams of Alanyl-Glutamine. In another specific embodiment, a single dose of Alanyl-Glutamine (e.g., a total daily dose of Alanyl-Glutamine) comprises about 30-35 grams of Alanyl-Glutamine. In another specific embodiment, said dose of Alanyl-Glutamine as used in accordance with the methods is administered with 200-400 mg of zinc.

In another specific embodiment, a single dose of Alanyl-Glutamine (e.g., a total daily dose of Alanyl-Glutamine) comprises about 35 grams of Alanyl-Glutamine. In another specific embodiment, a single dose of Alanyl-Glutamine (e.g., a total daily dose of Alanyl-Glutamine) comprises about 35-40 grams of Alanyl-Glutamine. In another specific embodiment, said dose of Alanyl-Glutamine as used in accordance with the methods is administered with 200-400 mg of zinc.

In another specific embodiment, a single dose of Alanyl-Glutamine (e.g., a total daily dose of Alanyl-Glutamine) comprises about 40 grams of Alanyl-Glutamine. In another specific embodiment, a single dose of Alanyl-Glutamine (e.g., a total daily dose of Alanyl-Glutamine) comprises about 40-45 grams of Alanyl-Glutamine. In another specific embodiment, said dose of Alanyl-Glutamine as used in accordance with the methods is administered with 200-400 mg of zinc.

In another specific embodiment, a single dose of Alanyl-Glutamine (e.g., a total daily dose of Alanyl-Glutamine) comprises about 45 grams of Alanyl-Glutamine. In another specific embodiment, a single dose of Alanyl-Glutamine (e.g., a total daily dose of Alanyl-Glutamine) comprises about 45-50 grams of Alanyl-Glutamine. In another specific embodiment, said dose of Alanyl-Glutamine as used in accordance with the methods is administered with 200-400 mg of zinc.

In another specific embodiment, a single dose of Alanyl-Glutamine (e.g., a total daily dose of Alanyl-Glutamine) comprises about 50 grams of Alanyl-Glutamine. In another specific embodiment, a single dose of Alanyl-Glutamine (e.g., a total daily dose of Alanyl-Glutamine) comprises about 50-55 grams of Alanyl-Glutamine. In another specific embodiment, said dose of Alanyl-Glutamine as used in accordance with the methods is administered with 200-400 mg of zinc.

In another specific embodiment, a single dose of Alanyl-Glutamine (e.g., a total daily dose of Alanyl-Glutamine) comprises about 55 grams of Alanyl-Glutamine. In another specific embodiment, a single dose of Alanyl-Glutamine (e.g., a total daily dose of Alanyl-Glutamine) comprises about 55-60 grams of Alanyl-Glutamine. In another specific embodiment, said dose of Alanyl-Glutamine as used in accordance with the methods is administered with 200-400 mg of zinc.

In another specific embodiment, a single dose of Alanyl-Glutamine (e.g., a total daily dose of Alanyl-Glutamine) comprises about 60 grams of Alanyl-Glutamine. In another specific embodiment, a single dose of Alanyl-Glutamine (e.g., a total daily dose of Alanyl-Glutamine) comprises about 60-65 grams of Alanyl-Glutamine. In another specific embodiment, said dose of Alanyl-Glutamine as used in accordance with the methods is administered with 200-400 mg of zinc.

In certain embodiments, a dose of Alanyl-Glutamine (e.g., a total daily dose of Alanyl-Glutamine) as described herein is formulated such that, when administered, it results in an increased level of glutamine in the body (e.g., in the tissues (e.g., mucosal lining of the mouth), blood, or plasma) of a subject receiving the dose as compared to the level of glutamine in the body achieved by administration of an equivalent dose of glutamine. See Section 7.2.

### 5.2.2 Modes of Administration

The Alanyl-Glutamine formulations provided herein are formulated for oral administration. In a specific embodiment, the Alanyl-Glutamine formulations provided herein are administered in the form of a liquid, e.g., a beverage, that can be readily consumed by a patient in need thereof, e.g., a patient undergoing a cancer treatment or that is scheduled to undergo a cancer treatment in the future. In certain embodiments, Alanyl-Glutamine formulations provided herein are orally administered in non-liquid form, e g., are frozen or formulated as a gel or goo.

### 5.2.3 Patient Populations

The patients treated in accordance with the methods described herein are undergoing cancer treatment or expected to undergo cancer treatment (and are administered an Alanyl-Glutamine formulation before (and after) the treatment commences). In a specific embodiment, the patient is undergoing or expected to undergo chemotherapy treatment, e.g., high dose chemotherapy treatment. In another specific embodiment, the patient is undergoing or expected to undergo radiation treatment, e.g., altered fractionation radiation treatment. In another specific embodiment, the patient is undergoing or expected to undergo hematopoietic stem cell transplant. In a preferred embodiment the patient in need thereof is being treated with high dose chemotherapy. Cancer treatments are discussed in Section 5.2.5.

The patients treated in accordance with the methods described herein can have or be expected to develop one or more cancer treatment side effects.

In a specific embodiment, a patient treated in accordance with the methods described herein has been diagnosed with oral mucositis resulting from cancer treatment. In another specific embodiment, a patient treated in accordance with the methods described herein is expected to develop oral mucositis based on a cancer treatment they are expected to receive. See Section 5.2.3.1.

In a specific embodiment, a patient treated in accordance with the methods described herein has been diagnosed with peripheral neuropathy resulting from cancer treatment. In another specific embodiment, a patient treated in accordance with the methods described herein is expected to develop peripheral neuropathy based on a cancer treatment they are expected to receive. See Section 5.2.3.2.

In a specific embodiment, a patient treated in accordance with the methods described herein has been diagnosed with cardiac damage (e.g., cardiomyopathy) resulting from cancer treatment. In another specific embodiment, a patient treated in accordance with the methods described herein is expected to develop cardiac damage (e g, cardiomyopathy) based on a cancer treatment they are expected to receive. See Section 5.2.3.3.

In a specific embodiment, a patient treated in accordance with the methods described herein has been diagnosed with dysgeusia resulting from cancer treatment. In another specific embodiment, a patient treated in accordance with the methods described herein is expected to develop dysgeusia based on a cancer treatment they are expected to receive. See Section 5.2.3.4.

In a specific embodiment, a patient treated in accordance with the methods described herein has been diagnosed with hypertension resulting from cancer treatment. In another specific embodiment, a patient treated in accordance with the methods described herein is expected to develop hypertension based on a cancer treatment they are expected to receive. See Section 5.2.3.5.

In certain embodiments, the patients treated in accordance with the methods described herein are undergoing cancer treatment or expected to undergo cancer treatment (and are administered an Alanyl-Glutamine formulation before (and after) the treatment commences), but do not have cancer and instead have been diagnosed with a disease/condition for which treatment with a cancer treatment is indicated. For example, and without limitation, a patient treated in accordance with the methods described herein can be undergoing cancer treatment or expected to undergo cancer treatment for an autoimmune condition (e.g., chemotherapy treatment), an inflammatory condition (e.g., chemotherapy treatment), neutropenia (e.g., treatment with cyclosporine), rheumatoid arthritis (e.g., treatment with methotrexate), multiple sclerosis (e.g., treatment with autologous stem cell transplant), or lupus erythematosus (e.g., treatment with Rituximab).

In certain embodiments, the patients treated in accordance with the methods described herein are orally administered a dose of Alanyl-Glutamine (e.g., a total daily dose of Alanyl-Glutamine) that results in an increased level of glutamine in the body (e.g., in the tissues (e.g., mucosal lining of the mouth), blood, or plasma) of the patients receiving the dose as compared to the level of glutamine in the body achieved by administration of an equivalent dose of glutamine. See Section 7.2.

### 5.2.3.1 Oral Mucositis

Oral mucositis (OM) is a common toxicity of both chemotherapy and radiation, and is caused by damage to the oral and/or intestinal mucosa. Considered one of the most acute side effects of cancer treatment, OM can cause redness, swelling, bleeding and sores in the mouth and throughout the entire digestive tract, resulting in severe oral pain and difficulty swallowing, eating, and drinking. Oral mucositis can also limit the ability of the epithelial lining to absorb nutrients from the diet, which in combination with the inability or lack of interest in eating, can lead to poor nutrition, weight loss, and cancer cachexia. Furthermore, ulcerations in the mouth and intestinal lining that present with oral mucositis can facilitate bacterial translocation leading to an increased incidence of infection. Oral mucositis can become increasingly severe leading to the hospitalization of patients undergoing cancer treatment and interruption of cancer treatment, leading to suboptimal treatment outcomes. The incidence of oral mucositis is typically between 20-40% in cancer patients undergoing treatment, however, this number can greatly increase, approaching 50% in patients receiving altered fractionation radiation and nearly 100% in patients undergoing treatment for hematologic malignancies (particularly in preparation for bone marrow transplantation), head and neck malignancies, as well as high-dose chemotherapy and radiation. Moreover, oral mucositis can occur in patients that are undergoing combination therapy for their cancer treatment, including the combination of chemotherapy and radiation. Oral mucositis may also occur in patients receiving targeted therapy and/or biologic therapy, whether administered as a single agent or in combination with chemotherapy or radiation.

Oral mucositis is a dose-limiting toxicity (DLT), whereby severe mucositis can result in chemotherapy or radiation dose modification, treatment duration reduction, or delay of administration of a dose or cycle of therapy while the mucositis resolves or decreases in severity. These decrease the overall treatment exposure for the patient and consequently lead to a less effective regimen and an increased possibility of treatment failure or tumor relapse. Accordingly, prevention or amelioration of oral mucositis can decrease patient discomfort and pain and allow more patients to complete their full course of treatment to achieve maximal effectiveness, tumor response, and consequently increase overall survival.

In a specific embodiment, a patient treated in accordance with the methods described herein has been diagnosed with oral mucositis resulting from cancer treatment. In another specific embodiment, a patient treated in accordance with the methods described herein is expected to develop oral mucositis based on a cancer treatment they are expected to receive.

In a specific embodiment, a patient with oral mucositis (or expected to develop oral mucositis) treated in accordance with the methods described herein has been diagnosed with head and neck cancer, and is undergoing a cancer treatment indicated for treatment of head and neck cancer.

In another specific embodiment, a patient with oral mucositis (or expected to develop oral mucositis) treated in accordance with the methods described herein has been diagnosed with breast cancer, and is undergoing a cancer treatment indicated for treatment of breast cancer.

In another specific embodiment, a patient with oral mucositis (or expected to develop oral mucositis) treated in accordance with the methods described herein has been diagnosed with colon cancer.

In another specific embodiment, a patient with oral mucositis (or expected to develop oral mucositis) treated in accordance with the methods described herein has been diagnosed with lung cancer, and is undergoing a cancer treatment indicated for treatment of lung cancer.

In certain embodiments, administration of an Alanyl-Glutamine formulation described herein to a patient results in prevention of oral mucositis, i.e., the patient undergoing cancer treatment never develops oral mucositis.

In certain embodiments, administration of an Alanyl-Glutamine formulation described herein to a patient results in a reduction in the duration of oral mucositis, e.g., a reduction in the total number of days the patient has oral mucositis.

In certain embodiments, administration of an Alanyl-Glutamine formulation described herein to a patient results in a reduction in the severity of oral mucositis. A reduction in severity can be indicated by a reduction in the most severe grade of mucositis achieved in a patient, the total number of days at the most severe grade of mucositis, or the overall number of days with grade 1 or greater mucositis.

In certain embodiments, administration of an Alanyl-Glutamine formulation described herein to a patient with oral mucositis results in an improved ability of the patient to ingest food. In certain embodiments, administration of an Alanyl-Glutamine formulation described herein to a patient with oral mucositis results in an improved ability of the patient to ingest water.

In certain embodiments, administration of an Alanyl-Glutamine formulation described herein to a patient with oral mucositis results in reduced patient weight-loss.

In certain embodiments, administration of an Alanyl-Glutamine formulation described herein to a patient with oral mucositis results in a reduction in the use of TPN (total parenteral nutrition) due to severe mucositis. In certain embodiments the regimen results in improved patient reported outcomes (PROs).

In certain embodiments, administration of an Alanyl-Glutamine formulation described herein to a patient with oral mucositis results in a reduction in the incidence of radiation dosage reduction due to mucositis, e.g., a 5, 10, 15, 20, 25% or greater reduction. In certain embodiments, administration of an Alanyl-Glutamine formulation described herein to a patient with oral mucositis results in a reduction in the incidence of radiation discontinuation due to mucositis. In certain embodiments, administration of an Alanyl-Glutamine formulation described herein to a patient with oral mucositis results in a reduction in the incidence of dosage skipping due to mucositis.

In certain embodiments, administration of an Alanyl-Glutamine formulation described herein to a patient with oral mucositis results in a reduction in the duration and/or dose and/or use of opiates by the patient.

In certain embodiments, administration of an Alanyl-Glutamine formulation described herein to a patient with oral mucositis results in a reduction in the duration and/or frequency of hospitalization of the patient.

In certain embodiments, administration of an Alanyl-Glutamine formulation described herein to a patient with oral mucositis results in a reduction in the rate and/or incidence and/or duration of infections in the patient.

In certain embodiments, administration of an Alanyl-Glutamine formulation described herein to a patient with oral mucositis results in an increase in progression free survival of the patient.

In certain embodiments, administration of an Alanyl-Glutamine formulation described herein to a patient with oral mucositis results in an increase in overall survival of the patient.

In certain embodiments, administration of an Alanyl-Glutamine formulation described herein to a patient with oral mucositis results in an increase in the likelihood that the patient will have a complete response (CR) or a partial response (PR) to their cancer treatment and/or decreases the time to CR or PR.

In certain embodiments, administration of an Alanyl-Glutamine formulation described herein to a patient with oral mucositis results in an increase in the time to progression (TTP). In certain embodiments, administration of an Alanyl-Glutamine formulation described herein to a patient with oral mucositis results in an improvement in the objective response (OR).

### 5.2.3.2 Peripheral Neuropathy

Chemotherapy-induced peripheral neuropathy (CIPN or PN) is another common dose-limiting toxicity associated with several commonly used cancer treatments including, but not limited, to Bortezomib, Platinums (and other platinum-based chemotherapies, compounds, or regimens), Taxanes, Thalidomide, Vinca alkaloids, and radiation therapy. Peripheral neuropathy is characterized by paresthesia, dysesthesia, impaired movement and pain. Patients suffering from peripheral neuropathy experience damage to the peripheral nerves causing sensory symptoms in the hands and the feet, which commonly manifest as numbness, tingling, burning, throbbing, stabbing sensations, and/or pain. Chemotherapy-induced peripheral neuropathy (CIPN) can be extremely painful and disabling, causing a significant loss of functional abilities. The symptoms can become more severe over multiple treatment cycles and are often strongly correlated with the cumulative dose of chemotherapy and/or radiation. Although symptoms generally resolve following the completion of treatment, in some patients they can persist for years after treatment is terminated. Moreover, it has been shown that in a third of CIPN cases the side effects have been found to be permanent. The incidence of peripheral neuropathy is variable, with 30-85% of patients receiving chemotherapy experiencing some degree of CIPN. This variability is largely due to the type of chemotherapy administered.

Chemotherapy-induced peripheral neuropathy can become so severe and painful that it becomes dose-limiting whereby the patients must undergo dose-reduction, treatment discontinuation, or delay of a dose or cycle of therapy. This results in a less effective treatment regime and leaves patients with suboptimal efficacy and an increased likelihood of tumor recurrence due to the failure to adequately eradicate cancer cells. Accordingly, the prevention or amelioration of CIPN can decrease patient discomfort and pain and allow more patients to complete their full course of treatment to achieve maximal tumor response and consequently increase overall survival.

In a specific embodiment, a patient treated in accordance with the methods described herein has been diagnosed with peripheral neuropathy resulting from cancer treatment. In another specific embodiment, a patient treated in accordance with the methods described herein is expected to develop peripheral neuropathy based on a cancer treatment they are expected to receive.

In a specific embodiment, a patient with peripheral neuropathy (or expected to develop peripheral neuropathy) is undergoing or is expected to undergo treatment with chemotherapy. In a specific embodiment, a patient with peripheral neuropathy (or expected to develop peripheral neuropathy) is undergoing or is expected to undergo treatment with Bortezomib, a platinum, a taxane, Thalidomide, and/or a vinca alkaloid.

In a specific embodiment, a patient with peripheral neuropathy (or expected to develop peripheral neuropathy) is undergoing or is expected to undergo treatment with radiation.

In certain embodiments, administration of an Alanyl-Glutamine formulation described herein to a patient results in prevention of peripheral neuropathy, i.e., the patient undergoing cancer treatment never develops peripheral neuropathy.

In certain embodiments, administration of an Alanyl-Glutamine formulation described herein to a patient results in a reduction in the duration of peripheral neuropathy, e.g., a reduction in the total number of days the patient has peripheral neuropathy.

In certain embodiments, administration of an Alanyl-Glutamine formulation described herein to a patient results in a reduction in the severity of peripheral neuropathy. A reduction in severity can be indicated by a reduction in the most severe grade of peripheral neuropathy achieved in a patient, the total number of days at the most severe grade of peripheral neuropathy, or the overall number of days with grade 1 or greater peripheral neuropathy.

In certain embodiments, administration of an Alanyl-Glutamine formulation described herein to a patient with peripheral neuropathy results in a reduction in weakness in the patient's extremities.

In certain embodiments, administration of an Alanyl-Glutamine formulation described herein to a patient with peripheral neuropathy results in a reduction in motor weakness.

In certain embodiments, administration of an Alanyl-Glutamine formulation described herein to a patient with peripheral neuropathy results in a reduction in numbness in the patient's fingers and/or toes.

In certain embodiments, administration of an Alanyl-Glutamine formulation described herein to a patient with peripheral neuropathy results in a reduction in tingling sensations experienced by the patient.

In certain embodiments, administration of an Alanyl-Glutamine formulation described herein to a patient with peripheral neuropathy results in a reduction in paresthesias and/or a reduction in dysaesthesias.

In certain embodiments, administration of an Alanyl-Glutamine formulation described herein to a patient with peripheral neuropathy results in a reduction in pain experienced by the patient.

In certain embodiments, administration of an Alanyl-Glutamine formulation described herein to a patient with peripheral neuropathy results in a reduction in severity of nerve injury in the patient.

In certain embodiments, administration of an Alanyl-Glutamine formulation described herein to a patient with peripheral neuropathy results in a reduction in the loss of vibratory sensation.

In certain embodiments, administration of an Alanyl-Glutamine formulation described herein to a patient with peripheral neuropathy results in a reduction of compound motor action potential (CAMP).

In certain embodiments, administration of an Alanyl-Glutamine formulation described herein to a patient with peripheral neuropathy results in a reduction of sensory nerve action potential (SNAP).

In certain embodiments, administration of an Alanyl-Glutamine formulation described herein to a patient with peripheral neuropathy results in a reduction in the duration and/or frequency of hospitalization of the patient.

In certain embodiments, administration of an Alanyl-Glutamine formulation described herein to a patient with peripheral neuropathy results in an increase in progression free survival of the patient.

In certain embodiments, administration of an Alanyl-Glutamine formulation described herein to a patient with peripheral neuropathy results in an increase in overall survival of the patient.

In certain embodiments, administration of an Alanyl-Glutamine formulation described herein to a patient with peripheral neuropathy results in an increase in the likelihood that the patient will have a complete response (CR) or a partial response (PR) to their cancer treatment and/or decreases the time to CR or PR.

In certain embodiments, administration of an Alanyl-Glutamine formulation described herein to a patient with peripheral neuropathy results in an increase in the time to progression (TTP). In certain embodiments, administration of an Alanyl-Glutamine formulation described herein to a patient with peripheral neuropathy results in an improvement in the objective response (OR).

### 5.2.3.3 Cardiac Damage

Cardiovascular toxicity is another potential short- or long-term side effect of cancer treatments such as radiation and chemotherapy. Cardiac damage (also known as chemotherapy related cardiac dysfunction (CRCD) and chemotherapy induced cardiomyopathy (CIC)), can be induced by numerous different chemotherapies including, but not limited to, anthracyclines, alkylating agents, taxols, monoclonal antibodies such as Herceptin, Bevacizumab, tyrosine kinase inhibitors, antimetabolites, arsenic trioxide and Tamoxifen. Exposure to these therapies can cause clinically significant cardiac dysfunction which can persist after treatment completion and in some cases can be irreversible and lead to heart failure and death. Cardiac toxicities associated with cancer treatment range from asymptomatic subclinical abnormalities such as electrocardiagraphic changes to potentially fatal conditions such as congestive heart failure and acute coronary syndromes. Cardiac dysfunction has been strongly correlated to cumulative dose for some chemotherapies including but not limited to anthracycline, while others such as trastuzumab are not associated with total dose or dosing frequency.

Many chemotherapeutic agents are associated with a high incidence of cardiac toxicities. The potential for long-term cardiac damage, particularly in high risk patient populations can lead to modified chemotherapy and radiation dosage whereby the patients receive reduced therapy or treatments are delayed or skipped. However, some patients may develop cardiac toxicities that can impact short and long-tem health, overall survival, and quality of life. Accordingly, a method to ameliorate chemotherapy and radiation induced cardiac damage can allow patients to receive maximum cancer therapy and represent an improvement to overall health outcomes and survival for cancer patients.

In a specific embodiment, a patient treated in accordance with the methods described herein has been diagnosed with cardiac damage (e.g., cardiomyopathy) resulting from cancer treatment. In another specific embodiment, a patient treated in accordance with the methods described herein is expected to develop cardiac damage (e.g., cardiomyopathy) based on a cancer treatment they are expected to receive.

In a specific embodiment, a patient with cardiac damage (e.g., cardiomyopathy) (or expected to develop cardiac damage) is undergoing or is expected to undergo treatment with chemotherapy. In a specific embodiment, a patient with cardiac damage (e.g., cardiomyopathy) (or expected to develop cardiac damage) is undergoing or is expected to undergo treatment with an anthracycline, an alkylating agent, a taxol, a monoclonal antibody (such as Herceptin or Bevacizumab), a tyrosine kinase inhibitor, an antimetabolite, arsenic trioxide, and/or Tamoxifen.

In a specific embodiment, a patient with cardiac damage (e.g., cardiomyopathy) (or expected to develop cardiac damage) is undergoing or is expected to undergo treatment with radiation.

In certain embodiments, administration of an Alanyl-Glutamine formulation described herein to a patient results in prevention of cardiac damage (e.g., cardiomyopathy), i.e., the patient undergoing cancer treatment never develops cardiac damage (e.g, cardiomyopathy).

In certain embodiments, administration of an Alanyl-Glutamine formulation described herein to a patient results in a reduction in the severity of cardiac damage (e.g., cardiomyopathy).

In certain embodiments, administration of an Alanyl-Glutamine formulation described herein to a patient with cardiac damage (e.g., cardiomyopathy) results in a reduction in myocardial dysfunction.

In certain embodiments, administration of an Alanyl-Glutamine formulation described herein to a patient with cardiac damage (e.g., cardiomyopathy) results in a reduction a reduction in the incidence of heart failure.

In certain embodiments, administration of an Alanyl-Glutamine formulation described herein to a patient with cardiac damage (e.g., cardiomyopathy) results in improved ventricular ejection fraction.

In certain embodiments, administration of an Alanyl-Glutamine formulation described herein to a patient with cardiac damage (e.g., cardiomyopathy) results in improved fractional shortening.

In certain embodiments, administration of an Alanyl-Glutamine formulation described herein to a patient with cardiac damage (e.g., cardiomyopathy) results in a reduction in ventricular (LV) ventricular dysfunction.

In certain embodiments, administration of an Alanyl-Glutamine formulation described herein to a patient with cardiac damage (e.g., cardiomyopathy) results in a reduction in thrombosis.

In certain embodiments, administration of an Alanyl-Glutamine formulation described herein to a patient with cardiac damage (e.g., cardiomyopathy) results in a reduction in the incidence of arrhythmia.

In certain embodiments, administration of an Alanyl-Glutamine formulation described herein to a patient with cardiac damage (e.g., cardiomyopathy) results in a reduction in the incidence of abnormalities in ventricular repolarization.

In certain embodiments, administration of an Alanyl-Glutamine formulation described herein to a patient with cardiac damage (e.g., cardiomyopathy) results in a reduction of abnormalities in QT function.

In certain embodiments, administration of an Alanyl-Glutamine formulation described herein to a patient with cardiac damage (e.g., cardiomyopathy) results in a reduction in acute coronary syndromes.

In certain embodiments, administration of an Alanyl-Glutamine formulation described herein to a patient with cardiac damage (e.g., cardiomyopathy) results in a reduction in systolic or diastolic cardiac dysfunction.

In certain embodiments, administration of an Alanyl-Glutamine formulation described herein to a patient with cardiac damage (e.g., cardiomyopathy) results in an increase in progression free survival of the patient.

In certain embodiments, administration of an Alanyl-Glutamine formulation described herein to a patient with cardiac damage (e.g., cardiomyopathy) results in an increase in overall survival of the patient.

In certain embodiments, administration of an Alanyl-Glutamine formulation described herein to a patient with cardiac damage (e.g., cardiomyopathy) results in an increase in the likelihood that the patient will have a complete response (CR) or a partial response (PR) to their cancer treatment and/or decreases the time to CR or PR.

In certain embodiments, administration of an Alanyl-Glutamine formulation described herein to a patient with cardiac damage (e.g., cardiomyopathy) results in an increase in the time to progression (TTP). In certain embodiments, administration of an Alanyl-Glutamine formulation described herein to a patient with cardiac damage (e.g., cardiomyopathy) results in an improvement in the objective response (OR).

### 5.2.3.4 Dysgeusia (and Related Conditions)

Patients undergoing chemotherapy and radiation often develop dysgeusia, altered taste acuity, due to the toxic side-effects of treatment. Dysgeusia occurs in up to 77% of cancer patients being treated with chemotherapy including, but not limited to, cyclophosphamide, dacarbazine, doxorubicin, 5-FU, methotrexate, cisplatin, and vincristine. The most common changes associated with dysgeusia include, but are not limited to, sweet, sour, salt, bitter, and/or savory/umami taste changes. Other gustatory conditions related to taste dysfunction include the increased sensitivity threshold to taste perception (Hypogeusia), the absence of taste perception (Ageusia), the perception of metallic or salty tastes without an external stimulus (Phantogeusia). In addition, the physiological changes to taste-mediating cells causing altered taste perception is further compounded by similar damage to olfactory senses due to the strong correlation between odor and taste in overall flavor recognition. The occurrence and severity of dysgeusia in cancer patients severely impacts their quality of life (QOL) and has been shown to lead to food aversion, weight-loss, malnutrition, cancer cachexia and anorexia. Improper or compromised nutrition in cancer patients and weight-loss have been strongly correlated with negative outcomes including prolonged morbidity, higher risk of infection, increased hospitalization, decreased overall survival as well as other clinical and survival related endpoints. Although dysgeusia is a common side-effect of treatment that significantly limits quality of life and contributes to poor nutritional status and impaired response to therapy, it is rarely addressed in a clinical setting. Currently the most common management technique is modified food preparation based on the patients taste alterations, however, prevention or amelioration of dysgeusia would increase the patient's ability to ingest food and liquids and help them maintain adequate nutrition throughout treatment. This would prevent malnutrition and help maintain a healthy immune system and improve overall patient outcomes.

In a specific embodiment, a patient treated in accordance with the methods described herein has been diagnosed with dysgeusia and/or hypogeusia and/or ageusia and/or phantogeusia resulting from cancer treatment. In another specific embodiment, a patient treated in accordance with the methods described herein is expected to develop dysgeusia and/or hypogeusia and/or ageusia and/or phantogeusia based on a cancer treatment they are expected to receive.

In a specific embodiment, a patient with dysgeusia and/or hypogeusia and/or ageusia and/or phantogeusia (or expected to develop dysgeusia and/or hypogeusia and/or ageusia and/or phantogeusia) is undergoing or is expected to undergo treatment with chemotherapy. In a specific embodiment, a patient with dysgeusia and/or hypogeusia and/or ageusia and/or phantogeusia (or expected to develop dysgeusia and/or hypogeusia and/or ageusia and/or phantogeusia) is undergoing or is expected to undergo treatment with cyclophosphamide, dacarbazine, doxorubicin, 5-FU, methotrexate, cisplatin, and/or vincristine.

In a specific embodiment, a patient with dysgeusia and/or hypogeusia and/or ageusia and/or phantogeusia (or expected to develop dysgeusia and/or hypogeusia and/or ageusia and/or phantogeusia) is undergoing or is expected to undergo treatment with radiation.

In certain embodiments, administration of an Alanyl-Glutamine formulation described herein to a patient results in prevention of dysgeusia and/or hypogeusia and/or ageusia and/or phantogeusia (or expected to develop dysgeusia and/or hypogeusia and/or ageusia and/or phantogeusia), i.e., the patient undergoing cancer treatment never develops dysgeusia and/or hypogeusia and/or ageusia and/or phantogeusia (or expected to develop dysgeusia and/or hypogeusia and/or ageusia and/or phantogeusia).

In certain embodiments, administration of an Alanyl-Glutamine formulation described herein to a patient results in a reduction in the duration of dysgeusia and/or hypogeusia and/or ageusia and/or phantogeusia (or expected to develop dysgeusia and/or hypogeusia and/or ageusia and/or phantogeusia), e.g., a reduction in the total number of days the patient has dysgeusia and/or hypogeusia and/or ageusia and/or phantogeusia (or expected to develop dysgeusia and/or hypogeusia and/or ageusia and/or phantogeusia).

In certain embodiments, administration of an Alanyl-Glutamine formulation described herein to a patient with peripheral neuropathy results in a reduction in increased sensitivity to taste perception (hyogeusia), reduction in the absence of taste perception (Ageusia), reduction in the perception of metallic or salty tastes without an external stimulus (Phantogeusia), reduction in the decreased sensitivity to odor perception (Hyposmia), reduction in the distorted ability to identify odors (Dysosmia), reduction in the inability to discriminate perceived odors (Agnosia), reduction in the altered odor perception in the presence of another odor (Parosmia), reduction in the odor perception without the presence of any odor (Phantosmia), and/or reduction in the absence of odor perception (Anosmia).

In certain embodiments, administration of an Alanyl-Glutamine formulation described herein to a patient with peripheral neuropathy results in a reduction in a lack of interest in eating.

In certain embodiments, administration of an Alanyl-Glutamine formulation described herein to a patient with dysgeusia and/or hypogeusia and/or ageusia and/or phantogeusia results in a reduction in numbness in the patient's fingers and/or toes.

In certain embodiments, administration of an Alanyl-Glutamine formulation described herein to a patient with dysgeusia and/or hypogeusia and/or ageusia and/or phantogeusia results in a reduction in weight loss by the patient.

In certain embodiments, administration of an Alanyl-Glutamine formulation described herein to a patient with dysgeusia and/or hypogeusia and/or ageusia and/or phantogeusia results in a reduction in the inability to eat due to taste alterations.

In certain embodiments, administration of an Alanyl-Glutamine formulation described herein to a patient with dysgeusia and/or hypogeusia and/or ageusia and/or phantogeusia results in a reduction in a disinterest in eating and drinking due to taste alterations.

In certain embodiments, administration of an Alanyl-Glutamine formulation described herein to a patient with dysgeusia and/or hypogeusia and/or ageusia and/or phantogeusia results in a quicker recovery of taste.

In certain embodiments, administration of an Alanyl-Glutamine formulation described herein to a patient with dysgeusia and/or hypogeusia and/or ageusia and/or phantogeusia results in a reduction in taste acuity.

In certain embodiments, administration of an Alanyl-Glutamine formulation described herein to a patient with dysgeusia and/or hypogeusia and/or ageusia and/or phantogeusia improved urea detection, sodium chloride detection, saccharose detection, and/or hydrogen chloride detection.

In certain embodiments, administration of an Alanyl-Glutamine formulation described herein to a patient with dysgeusia and/or hypogeusia and/or ageusia and/or phantogeusia results in a reduction in the incidence/severity of taste alterations (bitter, salty, sour, sweet, metallic).

In certain embodiments, administration of an Alanyl-Glutamine formulation described herein to a patient with dysgeusia and/or hypogeusia and/or ageusia and/or phantogeusia results in a reduction in the duration and/or frequency of hospitalization of the patient.

In certain embodiments, administration of an Alanyl-Glutamine formulation described herein to a patient with dysgeusia and/or hypogeusia and/or ageusia and/or phantogeusia results in an increase in progression free survival of the patient.

In certain embodiments, administration of an Alanyl-Glutamine formulation described herein to a patient with dysgeusia and/or hypogeusia and/or ageusia and/or phantogeusia results in an increase in overall survival of the patient.

In certain embodiments, administration of an Alanyl-Glutamine formulation described herein to a patient with dysgeusia and/or hypogeusia and/or ageusia and/or phantogeusia results in an increase in the likelihood that the patient will have a complete response (CR) or a partial response (PR) to their cancer treatment and/or decreases the time to CR or PR.

In certain embodiments, administration of an Alanyl-Glutamine formulation described herein to a patient with dysgeusia and/or hypogeusia and/or ageusia and/or phantogeusia results in an increase in the time to progression (TTP). In certain embodiments, administration of an Alanyl-Glutamine formulation described herein to a patient with dysgeusia and/or hypogeusia and/or ageusia and/or phantogeusia results in an improvement in the objective response (OR).

### 5.2.3.5 Hypertension

Cancer treatments, e.g., chemotherapy and radiation, can also cause hypertension in patients receiving them. Such hypertension can be deleterious to the health of cancer patients, and can represent a dose-limiting toxicity. Accordingly, the prevention or amelioration of hypertension can allow more patients to complete their full course of treatment to achieve maximal tumor response and consequently increase overall survival.

In a specific embodiment, a patient treated in accordance with the methods described herein has been diagnosed with hypertension resulting from cancer treatment. In another specific embodiment, a patient treated in accordance with the methods described herein is expected to develop hypertension based on a cancer treatment they are expected to receive.

In a specific embodiment, a patient with hypertension (or expected to develop hypertension) is undergoing or is expected to undergo treatment with chemotherapy.

In a specific embodiment, a patient with hypertension (or expected to develop hypertension) is undergoing or is expected to undergo treatment with radiation.

In certain embodiments, administration of an Alanyl-Glutamine formulation described herein to a patient results in prevention of hypertension, i.e., the patient undergoing cancer treatment never develops hypertension.

In certain embodiments, administration of an Alanyl-Glutamine formulation described herein to a patient results in a reduction in the duration of hypertension, e.g., a reduction in the total number of days the patient has hypertension.

In certain embodiments, administration of an Alanyl-Glutamine formulation described herein to a patient results in a reduction in the severity of hypertension.

In certain embodiments, administration of an Alanyl-Glutamine formulation described herein to a patient with hypertension results in a reduction in the duration and/or frequency of hospitalization of the patient.

In certain embodiments, administration of an Alanyl-Glutamine formulation described herein to a patient with hypertension results in an increase in progression free survival of the patient.

In certain embodiments, administration of an Alanyl-Glutamine formulation described herein to a patient with hypertension results in an increase in overall survival of the patient.

In certain embodiments, administration of an Alanyl-Glutamine formulation described herein to a patient with hypertension results in an increase in the likelihood that the patient will have a complete response (CR) or a partial response (PR) to their cancer treatment and/or decreases the time to CR or PR.

In certain embodiments, administration of an Alanyl-Glutamine formulation described herein to a patient with hypertension results in an increase in the time to progression (TTP). In certain embodiments, administration of an Alanyl-Glutamine formulation described herein to a patient with hypertension results in an improvement in the objective response (OR).

### 5.2.4 Types of Cancer

The methods described herein are relevant to patients diagnosed with all cancer types. Exemplary cancers that require cancer treatment that can result in cancer treatment side effects requiring treatment in accordance with the methods described herein include, without limitation, leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome, chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; dendritic cell cancer, including plasmacytoid dendritic cell cancer, NK blastic lymphoma (also known as cutaneous NK/T-cell lymphoma and agranular (CD4+/CD56+) dermatologic neoplasms); basophilic leukemia; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytoma and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

### 5.2.5 Cancer Treatments

The methods described herein are relevant to patients undergoing treatment with (or expected to be treated with) any type of cancer treatment that causes deleterious side effects. In a specific embodiment, the cancer treatment is chemotherapy. In a specific embodiment, the cancer treatment is radiation.

Non-limiting examples of cancer treatment include chemotherapy, radiotherapy, radioimmunotherapy, hormonal therapy, small molecule therapy, toxin therapy, pro-drug activating enzyme therapy, biologic therapy, antibody therapy, surgical therapy, immunotherapy, anti-angiogenic therapy, targeted therapy, epigenetic therapy, demethylation therapy, histone deacetylase inhibitor therapy, differentiation therapy, adjuvant therapy and/or any combination thereof.

Non-limiting examples of therapies with the potential to cause and/or induce oral mucositis include 5-fluorouracil, irinotecan, taxanes, folate-based drugs, methotrexate, etoposide anthracycline (breast cancer).

Non-limiting examples of therapies with the potential to cause and/or induce peripheral neuropathy include vinca alkaloids (vincristine), cisplatin, paclitaxel, podophyllotoxins (etoposide, tenoposide), thalidomide, interferon, taxanes, velcade, cytosine arabinoside, misonidazole.

Non-limiting examples of therapies with the potential to cause and/or induce dysgeusia and/or hypogeusia and/or ageusia and/or phantogeusia include cyclophosphamide, dacarbazine, doxorubicin, 5-FU, methotrexate, nitrogen mustard, cisplatin, and vincristine.

Non-limiting examples of therapies with the potential to cause and/or induce cardiac damage (e.g., cardiomyopathy) include anthracycline, alkylating agents, taxols, monoclonal antibodies such as Herceptin, Bevacizumab, tyrosine kinase inhibitors, antimetabolites, arsenic trioxide and Tamoxifen.

Non-limiting examples of therapies with the potential to cause and/or induce hypertension include VEGF inhibitors, VEGF receptor (VEGFR) inhibitors, bevacizumab, sorafenib, sunitinab, pazopanib, everolimus, and pontinib.

### 5.2.6 Combination Therapies

In certain embodiments, an Alanyl-Glutamine formulation described herein is a administered in combination with one or more additional therapies used in treatment of cancer treatment side effects.

In a specific embodiment, an Alanyl-Glutamine formulation described herein is administered in combination with a treatment for oral mucositis, wherein said treatment is one or more of a medical mouthwash (e.g., one containing saline, soda bicarbonate, calcium phosphate, antifungals, milk of magnesia and/or oral numbing agents such as lidocaine, or a combination thereof), Benzydamine, cryotherapy, oral analgesics, topical numbing agents (e.g., lidocaine, benzocaine, dyclonine hydrochloride (HCL) and Ulcerase®), and opioids (e.g., morphine and fentanyl), topical oral gel coating agents (e.g., Gelclair® and Zilactin®), antibiotics, anti-fungals, prophylactic low level laser therapy (LLLT), granulocyte-macrophage colony-stimulating factor (GM-CSF), pentoxifylline, topical prostaglandin E2 (PGE2), and Palifermin (Kepivance™).

In another specific embodiment, an Alanyl-Glutamine formulation described herein is administered in combination with a treatment for peripheral neuropathy, wherein said treatment is one or more of duloxetine (Cymbalta™), venlafaxine (Effexor™), Pregabalin (Lyrica™), Amifostine (Ethyol™), vitamin E, vitamin B6, omega-3 fatty acids, and acupuncture.

In another specific embodiment, an Alanyl-Glutamine formulation described herein is administered in combination with a treatment for cardiac damage (e.g., cardiomyopathy), wherein said treatment is one or more of enalapril (Vasotec®), valsartan (Diovan), telmisartan (ARB), a combination of β-blocker and angiotensin converting enzyme inhibitor (ACE-1).

### 6. Articles of Manufacture

Also provided herein are sealed and/or capped beverage containers comprising an Alanyl-Glutamine formulation described herein. In a specific embodiment, provided herein is a sealed and/or capped bottle comprising an Alanyl-Glutamine formulation described herein, wherein said bottle is capable of holding a volume of about or at least 0.5, 1, 2, 3, 4, 5, 6, 7, 8, 9, or 10 ounces. In another specific embodiment, the Alanyl-Glutamine formulation present in a sealed beverage container described herein comprises zinc.

In a specific embodiment, a sealed and/or capped beverage container comprising an Alanyl-Glutamine formulation described herein comprises a single dose (e.g., a total daily dose) of Alanyl-Glutamine. In another specific embodiment, a sealed and/or capped beverage container comprising an Alanyl-Glutamine formulation described herein comprises one-fifth, one-quarter, one-third, or one-half of a single dose (e.g., a total daily dose) of Alanyl-Glutamine. Doses of Alanyl-Glutamine are described in Section 5.2.1.

### 7. EXAMPLES

### 7.1 Example 1: Effect of Alanyl-Glutamine on Intestinal Crypt Survival

Chemotherapy and radiation therapy are designed to target rapidly proliferating tumors and as a consequence can adversely affect rapidly dividing cells in healthy tissue. One of the most rapidly dividing tissues of the body is the small intestine, which resultantly is a common site of toxicity during cancer therapy. Cancer treatments that result in the killing of crypt cells, the regenerative cells of the gut, lead to a breakdown of the mucosal barrier (ulceration). Problems associated with such damage, termed intestinal mucositis, can delay treatment or cause therapeutic doses to be reduced, impairing the success of cancer treatment. Treatments that may protect or spare more crypts during cancer therapy therefore improve patient well-being and may allow dose escalation and hence improve treatment outcomes.

In this example irradiation was used to target the proliferating clonogenic cells of the crypt. Following irradiation, crypt cells can become impaired and undergo apoptosis, or programmed cell death, resulting in a reduction in the number of viable crypts, which could regenerate and heal the intestinal tissue. The more crypts that are lost the greater the level of ulceration, which can manifest as the symptoms of mucositis. Agents that therefore reduce mucositis severity will also demonstrate improved crypt survival. This model of crypt cell survival is therefore often used as a surrogate for manifestation of mucositis, e.g., oral mucositis.

In this example the efficacy of orally administered Alanyl-Glutamine (Ala-Gln) and Glutamine (Gln) on small intestinal crypt survival and regeneration and maintenance of villus structure was assessed in response to radiation insult in a murine model. The crypt cells were measured at 4 days post high-dose irradiation, a time at which crypt cells affected by radiation have died and surviving cells begin to regenerate. The level of crypt cell survival at this time directly correlates to severity of ulceration and ultimately animal survival.

### Groups and Dosing

Ala-Gln and Gln were administered orally twice daily at a volume of 0.2 ml either pre- and post-irradiation (day −5 to day 4), or post-irradiation alone (day 0 to day 4). The animals were given doses of Ala-Gln at a concentration of 39.65 mg/ml and Gln at a concentration of 26.75 mg/ml, which contain molar equivalent doses of Gln. Vehicle control animals were administered 0.2 ml of water orally twice daily.

### Radiation and Dosimetry

Animals were irradiated on day 0 at 3 pm. Irradiation was performed using an XStrahl RS320 X-ray set, operated at 300 Kv, 10 Ma. Radiation was delivered at a dose rate of 0.832 Gy/min using a single uniform dose of 13 Gy radiation. A dosimetry device was placed in the irradiation chamber to confirm the dose received.

### Study Termination, Histological Processing and Crypt Survival Scoring

Animals were euthanized on day 4 post-irradiation. The proximal small intestine was fixed in Carnoy's fixative for 30-60 minutes and then transferred to 70% ethanol prior to processing to generate one paraffin embedded tissue block per four mice. The fixed small intestine was “bundled” prior to embedding in order to obtain the ideal orientation of the crypts. Each block was then sectioned and H&E stained to provide 1 H&E slide per block.

For each animal in the study, ten intestinal circumferences were analyzed, providing a total of 80 circumferences per treatment group available for analysis. The number of surviving and regenerating crypts per circumference was scored and the average per group was determined.

### Results/Conclusion

The number of surviving and regenerating crypts in all groups were compared. Groups treated pre-irradiation (−5 to ±4) contained significantly more crypts than groups that received treatment concurrent with radiation (0 to ±4). This result demonstrates that administering Ala-Gln and Gln prior to radiation reduces epithelial and crypt cell damage, allowing an increased number of crypts to survive. Of the two groups treated pre-irradiation (−5 to ±4), the group treated with Ala-Gln contained a higher average number of crypts that those treated with Gln. This indicates that Ala-Gln is more effective than Gln at preventing radiation-induced epithelial damage, thus preserving the largest number of crypts. Accordingly, this suggests that Ala-GIn may be more effective than Gln at preventing mucositis in patients through epithelial protection and reduction of ulceration.

### 7.2 Example 2: Alanyl-Glutamine Increases Plasma Glutamine Levels Greater than Glutamine In Vivo

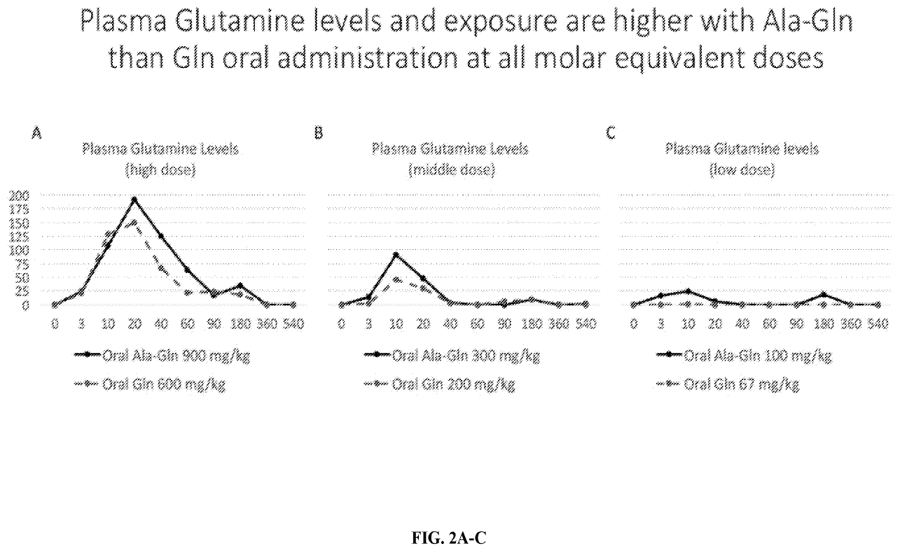

Alanyl-Glutamine and glutamine were administered as a single dose by oral gavage, with deionized water as the vehicle, into male Sprague Dawley rats. The oral gavage of Alanyl-Glutamine and glutamine was administered to different groups at three distinct levels, a high, medium and low dose. Each dose level contained molar equivalent amounts of Glutamine (doses selected to correspond to relevant human doses) so that the resulting changes in plasma Glutamine levels could be directly compared in vivo. See FIG. 2. The concurrent control group received the vehicle (deionized water) by oral gavage. The dose volume was 20 ml/kg for the oral gavage. Each treatment group consisted of three males. For pharmacokinetic evaluation, blood samples were collected via a jugular vein cannula (JVC) from 3 animals/group/time point prior to dose administration and at 3, 10, 20, 40, 60, 90, 180, 360, and 540 minutes after dose administration. Each sample was analyzed to determine plasma glutamine concentration using a qualified ultra-high performance liquid chromatography mass spectrometry method (UHPLC/MS/MS), developed expressly for the determination of glutamine levels in rat plasma. A subtractive analysis of the plasma levels was then performed, in which the data from each individual animal was subtracted from their pre-dose plasma glutamine levels. The resulting data-points were then averaged for each dosing group and graphed, and represent the change in plasma glutamine after administration of the two different compositions.

Following oral administration of Alanyl-Glutamine and glutamine, all animals showed an increase in glutamine levels in the plasma at all dose levels, as anticipated. See FIG. 2. However, an unexpected increase in plasma glutamine following oral administration of Alanyl-Glutamine was observed. See FIG. 2. In particular, despite being administered at equivalent molar doses, Alanyl-Glutamine administration resulted in higher plasma glutamine levels at each dose (high: FIG. 2A; middle: FIG. 2B; low: FIG. 2C) compared to plasma glutamine levels observed in rats administered glutamine. This surprising result indicates Alanyl-Glutamine is capable of increasing plasma Glutamine levels more efficiently and/or to a higher level than glutamine. This indicates that oral administration of Alanyl-Glutamine is preferable to glutamine administration.

### EQUIVALENTS

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. A composition formulated for oral administration, wherein said composition comprises a solution comprising Alanyl-Glutamine.

2. The composition of claim 1, wherein said solution comprises Alanyl-Glutamine and water.

3. The composition of claim 1, wherein said composition comprises about 0.5, 1, 1.5, 2, 2.5, 3, 3.5, 4, 4.5, 5, 5.5, or 6 ounces of solution.

4. The composition of claim 1, wherein said composition comprises no more than 0.5, 1, 1.5, 2, 2.5, 3, 3.5, 4, 4.5, 5, 5.5, or 6 ounces of solution.

5. The composition of claim 1, comprising about 1-5 grams of Alanyl-Glutamine/ounce of solution, about 5-10 grams of Alanyl-Glutamine/ounce of solution, about 10-15 grams of Alanyl-Glutamine/ounce of solution, about 15-20 grams of Alanyl-Glutamine/ounce of solution, about 20-25 grams of Alanyl-Glutamine/ounce of solution, about 25-30 grams of Alanyl-Glutamine/ounce of solution, about 30-35 grams of Alanyl-Glutamine/ounce of solution, about 35-40 grams of Alanyl-Glutamine/ounce of solution, about 40-45 grams of Alanyl-Glutamine/ounce of solution, about 45-50 grams of Alanyl-Glutamine/ounce of solution, about 50-55 grams of Alanyl-Glutamine/ounce of solution, about 55-60 grams of Alanyl-Glutamine/ounce of solution, about 60-65 grams of Alanyl-Glutamine/ounce of solution, about 65-70 grams of Alanyl-Glutamine/ounce of solution, about 70-75 grams of Alanyl-Glutamine/ounce of solution, about 75-80 grams of Alanyl-Glutamine/ounce of solution, about 80-85 grams of Alanyl-Glutamine/ounce of solution, about 85-90 grams of Alanyl-Glutamine/ounce of solution, about 90-95 grams of Alanyl-Glutamine/ounce of solution, or about 95-100 grams of Alanyl-Glutamine/ounce of solution.

6. The composition of claim 1, comprising zinc.

7. The composition of claim 6, comprising about 1-5 mg zinc/ounce of solution, about 5-10 mg zinc/ounce of solution, about 10-15 mg zinc/ounce of solution, about 15-20 mg zinc/ounce of solution, about 20-25 mg zinc/ounce of solution, about 25-30 mg zinc/ounce of solution, about 30-35 mg zinc/ounce of solution, about 35-40 mg zinc/ounce of solution, about 40-45 mg zinc/ounce of solution, about 45-50 mg zinc/ounce of solution, about 50-55 mg zinc/ounce of solution, about 55-60 mg zinc/ounce of solution, about 60-65 mg zinc/ounce of solution, about 65-70 mg zinc/ounce of solution, about 70-75 mg zinc/ounce of solution, about 75-80 mg zinc/ounce of solution, about 80-85 mg zinc/ounce of solution, about 85-90 mg zinc/ounce of solution, about 90-95 mg zinc/ounce of solution, about 95-100 mg zinc/ounce of solution, about 100-125 mg zinc/ounce of solution, about 125-150 mg zinc/ounce of solution, about 150-175 mg zinc/ounce of solution, about 175-200 mg zinc/ounce of solution, about 200-250 mg zinc/ounce of solution, about 250-300 mg zinc/ounce of solution, about 300-400 mg zinc/ounce of solution, or about 400-500 mg zinc/ounce of solution.

8. The composition of claim 1 comprising about 10-30 grams of Alanyl-Glutamine and about 100-400 mg of zinc in solution.

9. The composition of claim 1 comprising about 20-30 grams of Alanyl-Glutamine and about 100-400 mg of zinc in solution.

10. The composition of claim 1 comprising about 30-40 grams of Alanyl-Glutamine and about 100-400 mg of zinc in solution.

11. The composition of claim 1 comprising about 40-50 grams of Alanyl-Glutamine and about 100-400 mg of zinc in solution.

12. The composition of claim 1 comprising about 50-60 grams of Alanyl-Glutamine and about 100-400 mg of zinc in solution.

13. The composition of claim 1, comprising a total volume of about 0.5, 1, 1.5, 2, 2.5, 3, 3.5, 4, 4.5, 5, 5.5, or 6 ounces.

14. The composition of claim 1, comprising one or more of the following additional components: sweeteners (e.g., glucose, maltodextrin, maltose, dextrose, high fructose corn syrup, sucrose, lactose, fructose, Saccharin, aspartame, acesulfame K, sucralose, neotame, rebaudioside A, Stevia/Erythritol, Sorbitol, Mannitol, and Xylitol); thickening and/or gelling agents (e.g., alginate, propylene glycol alginate, carrageenans, cellulose gum and microcrystalline cellulose, Xanthum gums, and guar gums), coloring agents (e.g., Blue No. 2 (Indigotine), Red No. 40 (Allura Red AC), Yellow No. 5 (Tartrazine)); flavor and fragrance agents; agents capable of maintaining the formulations at a desired pH (e.g., citric acid, sodium citrate, zinc sulphase, magnesium sulphate, potassium chloride, and magnesium oxide); vitamins, e.g., vitamins A, D, E, K, B, C, Niacin, and folic acids); masking agents (e.g., citric acid, sodium citrates, sodium phosphate, and hydroxyl acids); and/or bitter taste-receptor blockers (e.g., hydrogenated ethoxylated glycerol ester, Beta-Cyclodextrin, homoeriodictyol sodium salt, and eriodictyol).

15. A container comprising the composition of claim 1.

16. The container of claim 15, wherein said container is a bottle, flask, tube, or jar.

17. A method for treating a cancer treatment side effect in a subject in need thereof, said method comprising oral administration of the composition of claim 1 to a patient suffering from a cancer treatment side effect.

18. (canceled)

19. The method of claim 17, wherein said cancer treatment side effect is one or more of oral mucositis, peripheral neuropathy, dysgeusia, cardiac damage, or hypertension.

20. The method of claim 17, wherein said cancer treatment side effect is caused by chemotherapy.

21. The method of claim 17, wherein said cancer treatment side effect is caused by radiation.

22. The method of claim 17, wherein said composition is administered prior to initiation of a cancer treatment of a subject.

23. The method of claim 22, wherein composition is administered 1, 2, 3, or 4 weeks or 1, 2, 3, 4, 5, 6, 7, 8, 9, 10, 11, 12, 13, 14, 15, 16, 17, 18, 19, 20, or 21 days prior to initiation of a cancer treatment regimen.

24. The method of claim 17, wherein said composition is administered after initiation of a cancer treatment of a subject.

25. The method of claim 24, wherein composition is administered 1, 2, 3, or 4 weeks or 1, 2, 3, 4, 5, 6, 7, 8, 9, 10, 11, 12, 13, 14, 15, 16, 17, 18, 19, 20, or 21 days after initiation of a cancer treatment regimen.

26. The method of claim 17, wherein said composition is administered concurrently with administration of a cancer treatment of a subject.

27. The method of claim 17, wherein said composition is administered once daily to the subject.

28. The method of claim 1, wherein said composition is administered more than once daily to the subject.

29. The method of claim 28, wherein said composition is administered two, three, four, five, or six times a day to the subject.

30. The method of claim 1, wherein said administration results in a higher plasma glutamine concentration than that observed following oral administration of an equivalent dose of glutamine.
